(19) United States
(12) Patent Application Publication
(10) Pub. No.: US 2026/0265352 A1
(43) Pub. Date: Sep. 10, 2026

(54) STABLE PHARMACEUTICAL FORMULATION COMPRISING ANTI-GREMLIN1 ANTIBODY

(71) Applicant: SUZHOU TRANSCENTA THERAPEUTICS CO., LTD., Suzhou, Jiangsu (CN)

(72) Inventors: Fan ZHANG, Suzhou, Jiangsu (CN); Pan MENG, Suzhou, Jiangsu (CN); Xueming QIAN, Suzhou, Jiangsu (CN); Di SUN, Suzhou, Jiangsu (CN); Hongjun LI, Suzhou, Jiangsu (CN); Huanhuan GUO, Suzhou, Jiangsu (CN)

(21) Appl. No.: 18/994,616

(22) PCT Filed: Jul. 18, 2023
(86) PCT No.: PCT/CN2023/107897

§ 371 Date: Jan. 15, 2025

(30) Foreign Application Priority Data
Jul. 18, 2022 (CN) 202210844602.X

(51) Int. Cl. C07K 16/18; A61P 35/00

(52) U.S. Cl. CPC C07K 16/18; A61P 35/00; C07K 2317/24; C07K 2317/565; C07K 2317/567; C07K 2317/92; C07K 2317/94

(57) ABSTRACT

Provided is a pharmaceutical formulation. The pharmaceutical formulation comprises an anti-GREM1 antibody, a buffer, a stabilizer, a surfactant, and optionally an antioxidant. The pharmaceutical formulation provided herein could maintain the stability of the anti-GREM1 antibody after long-term storage, storage at high temperature (e.g., 40° C.), storage at room temperature, vibration and/or multiple freezing and thawing cycles. Provided are the use of the pharmaceutical formulation in prevention and/or treatment of GREM1 related diseases, especially cancers (e.g., GREM1 related cancers), and a method for preparing the pharmaceutical formulation.

### FIELD OF THE INVENTION

The present disclosure relates to a pharmaceutical formulation, and in particular, relates to a stable pharmaceutical formulation comprising an anti-gremlin1 (GREM1) antibody. The present disclosure also relates to a method of preparing the pharmaceutical formulation and uses thereof.

### BACKGROUND

Gremlin1 (GREM1) is a highly conserved secretory protein with a cysteine-rich region and a cysteine junction (Wordinger et al., Experimental Eye Research (Exp Eye Res.), August 2008; 87(2): 78-79.). As a member of the differential screening-selected gene aberrative in neuroblastoma (DAN), the GREM1 is used as an antagonist of a bone morphogenetic protein (BMP) (Wordinger et al., Exp Eye Res., August 2008; 87(2): 78-79.). The GREM1 can physically bind to BMP-2, BMP-4 or BMP-7 to form a heterodimer, prevent a BMP ligand from interacting with a corresponding BMP receptor and subsequently inhibit the activation of a BMP signaling pathway.

The GREM1 is closely related to fibrotic lesions of the kidney, lung, liver and retina as well as a variety of tumor types, including pancreatic cancer, colon cancer, lung cancer, glioma, gastric cancer and prostate cancer (Sneddon et al., Proceedings of the National Academy of Sciences (PNAS), October 2006; 103(40): 14842-14847). For example, through abnormal upregulation of the GREM1, colon cells outside a stem cell niche are endowed with tumorigenicity. It has also been found that the GREM1 is highly expressed and secreted by tumor stem cells to maintain stem cell properties in glioma (Yan, K. et al., Genes Development (Genes Dev), 28, 1085-1100 (2014)). Therefore, the GREM1 has been used as a therapeutic target in the treatment of GREM1 related diseases, such as the use of anti-GREM1 antibodies.

Since antibody molecules have complex multilevel protein structures and are prone to physical binding, which may cause undesirable immune responses, or may become unsafe for patients due to clogging the syringe or pumps during administration. Thus, one of the long-standing problems with liquid formulations of antibodies is stability issues due to aggregation.

Therefore, there is a need for pharmaceutical formulations of an anti-GREM1 antibodies having stability and consistent quality.

### SUMMARY

The present disclosure provides a stable pharmaceutical formulation comprising an anti-GREM1 antibody that remains homogeneous and stable after long-term storage, storage at high temperature e.g., 40° C.), storage at room temperature, vibration and/or multiple freezing and thawing cycles.

In one aspect, the present disclosure provides a pharmaceutical formulation, wherein the pharmaceutical formulation comprises an anti-GREM1 antibody and a buffer, and the pH value is 4.5-6.5.

In one aspect, the present disclosure provides a pharmaceutical formulation, wherein the pharmaceutical formulation comprises an anti-GREM1 antibody and a buffer, wherein the buffer is an acetate acid buffer or a histidine buffer, and the pH value is 5.5-6.5. In some embodiments, the concentration of the buffer in the pharmaceutical formulation is 5 mM-50 mM or 10 mM-30 mM.

In some embodiments, the pharmaceutical formulation further comprises a stabilizer.

In some embodiments, the concentration of the stabilizer in the pharmaceutical formulation is 0.5% (w/v)-20% (w/v) or 0.5% (w/v)-10% (w/v).

In some embodiments, the stabilizer is selected from the group consisting of: sucrose, trehalose, sorbitol, NaCl and arginine hydrochloride.

In some embodiments, the stabilizer is sucrose, wherein the concentration of the sucrose in the pharmaceutical formulation is 1% (w/v)-10% (w/v). In other embodiments, the stabilizer is arginine hydrochloride, wherein the concentration of the arginine hydrochloride in the pharmaceutical formulation is 1% (w/v)-5% (w/v). In other embodiments, the stabilizer is trehalose, and the concentration of the trehalose in the pharmaceutical formulation is 5% (w/v)-10% (w/v). In other embodiments, the stabilizer is sorbitol, and the concentration of the sorbitol in the pharmaceutical formulation is 2% (w/v)-8% (w/v). In other embodiments, the stabilizer is NaCl, and the concentration of the NaCl in the pharmaceutical formulation is 0.5% (w/v)-1.5% (w/v).

In some embodiments, the pharmaceutical formulation further comprises a surfactant.

In some embodiments, the concentration of the surfactant in the pharmaceutical formulation is 0.005% (w/v)-0.4% (w/v) or 0.01% (w/v)-0.2% (w/v).

In some embodiments, the surfactant is selected from the group consisting of: polysorbate 80 and polysorbate 20.

In some embodiments, the surfactant is polysorbate 80, wherein the concentration of the polysorbate 80 and/or the polysorbate 20 in the pharmaceutical formulation is 0.025% (w/v)-0.1% (w/v).

In some embodiments, the pharmaceutical formulation further comprises an antioxidant.

In some embodiments, the concentration of the antioxidant in the pharmaceutical formulation is 0.01% (w/v)-0.2% (w/v) or 0.02% (w/v)-0.06% (w/v).

In some embodiments, the antioxidant is selected from the group consisting of: methionine, cysteine, glutathione, sodium thiosulfate and ascorbic acid.

In some embodiments, the concentration of the anti-GREM1 antibody in the pharmaceutical formulation is 1 mg/ml-200 mg/ml.

In some embodiments, the concentration of the anti-GREM1 antibody in the pharmaceutical formulation is 20 mg/ml-40 mg/ml.

In some embodiments, the anti-GREM1 antibody comprises a heavy chain CDR1 (HCDR1) as set forth in SEQ ID NO: 1, an HCDR2 as set forth in SEQ ID NO: 2 and an HCDR3 as set forth in SEQ ID NO: 3, and/or a light chain CDR1 (LCDR1) as set forth in SEQ ID NO: 4, an LCDR2 as set forth in SEQ ID NO: 5 and an LCDR3 as set forth in SEQ ID NO: 6.

In some embodiments, the anti-GREM1 antibody comprises a CDR1, a CDR2 and a CDR3 of the heavy chain variable region as set forth in SEQ ID NO: 7, and/or a CDR1, a CDR2 and a CDR3 of the light chain variable region as set forth in SEQ ID NO: 8.

In some embodiments, the anti-GREM1 antibody comprises a heavy chain framework region 1 (HFR1) as set forth in QX₁QLVQSGSELKKPGASVKVSCKASGX₂TFT (SEQ ID NO: 24), an HFR2 as set forth in WMX₃QAPGQGLX₄WMG (SEQ ID NO: 25), an HFR3 as set forth in RFX₅FSLDTSVSTAYLQISSLKAEDTAVYYCAR (SEQ ID NO: 26) and an HFR4 as set forth in WGQGTMVTVSS (SEQ ID NO: 17), and/or a light chain framework region 1 (LFR1) as set forth in DVVMTQSPLSLPVTLGQPASISC (SEQ ID NO: 27), an LFR2 as set forth in WLQQRPGQSPRRLIX₆ (SEQ ID NO: 32), an LFR3 as set forth in GVPDRFSGSGSGTDFTLKISRVEAEDVGVYYC (SEQ ID NO: 29) and an LFR4 as set forth in FGQGTKLEIK (SEQ ID NO: 30), where X₁ is V or I; X₂ is Y or S; X₃ is R or K; X₄ is E or T; X₅ is V or A; and X₆ is Y or S.

In some embodiments, the anti-GREM1 antibody comprises a heavy chain framework region 1 (HFR1) as set forth in SEQ ID NO: 14, 18 or 21, an HFR2 as set forth in SEQ ID NO: 15, 19 or 22, an HFR3 as set forth in SEQ ID NO: 16, 20 or 23 and an HFR4 as set forth in SEQ ID NO: 17, and/or a light chain framework region 1 (LFR1) as set forth in SEQ ID NO: 27, an LFR2 as set forth in SEQ ID NO: 28 or 31, an LFR3 as set forth in SEQ ID NO: 29 and an LFR4 as set forth in SEQ ID NO: 30.

In some embodiments, the anti-GREM1 antibody comprises an FR1, an FR2, an FR3 and an FR4 of the heavy chain variable region as set forth in SEQ ID NO: 7, 11 or 12, and/or an FR1, an FR2, an FR3 and an FR4 of the light chain variable region as set forth in SEQ ID NO: 8 or 13.

In some embodiments, the anti-GREM1 antibody comprises a heavy chain variable region as set forth in SEQ ID NO: 7, 11 or 12, and/or a light chain variable region as set forth in SEQ ID NO: 8 or 13.

In some embodiments, the anti-GREM1 antibody comprises a heavy chain constant region as set forth in SEQ ID NO: 33, and/or a light chain constant region as set forth in SEQ ID NO: 34.

In some embodiments, the anti-GREM1 antibody comprises a heavy chain as set forth in SEQ ID NO: 9, and/or a light chain as set forth in SEQ ID NO: 10.

In some embodiments, the pharmaceutical formulation comprises an anti-GREM1 antibody, a buffer, a stabilizer and a surfactant, wherein the buffer is a histidine buffer, the stabilizer is arginine hydrochloride or sucrose, the surfactant is polysorbate 80 or polysorbate 20, and the pH value is about 4.5-6.5.

In some embodiments, the concentration of the anti-GREM1 antibody in the pharmaceutical formulation is 20 mg/ml-40 mg/ml, the concentration of the histidine buffer in the pharmaceutical formulation is 10 mM-30 mM, the concentration of the arginine hydrochloride or the sucrose in the pharmaceutical formulation is 1% (w/v)-10% (w/v), and/or the concentration of the polysorbate 80 or the polysorbate 20 in the pharmaceutical formulation is 0.025% (w/v)-0.1% (w/v).

In some embodiments, the concentration of the anti-GREM1 antibody in the pharmaceutical formulation is 33 mg/ml, the concentration of the histidine buffer in the pharmaceutical formulation is about 20 mM, the stabilizer is sucrose, wherein the concentration of the sucrose in the pharmaceutical formulation is 8% (w/v)-9% (w/v), the surfactant is polysorbate 80, wherein the concentration of the polysorbate 80 in the pharmaceutical formulation is about 0.05% (w/v), and the pH value is about 5.5-6.5.

In some embodiments, the pharmaceutical formulation further comprises an antioxidant, wherein the antioxidant is methionine. In some embodiments, the concentration of the methionine in the pharmaceutical formulation is about 0.04% (w/v).

In some embodiments, the concentration of the anti-GREM1 antibody in the pharmaceutical formulation is about 33 mg/ml, the concentration of the histidine buffer in the pharmaceutical formulation is about 20 mM, the stabilizer is sucrose, wherein the concentration of the sucrose in the pharmaceutical formulation is about 8.2% (w/v), the surfactant is polysorbate 80, the concentration of the polysorbate 80 in the pharmaceutical formulation is about 0.05% (w/v), the concentration of the methionine in the pharmaceutical formulation is about 0.04% (w/v), and the pH value is about 6.0.

In another aspect, the present disclosure provides use of the pharmaceutical formulation described herein in the manufacture of a medicament for preventing and/or treating GREM1 related diseases.

In some embodiments, the GREM1 related diseases are selected from the group consisting of: cancers, fibrosis diseases, angiogenesis, glaucoma or retinal diseases, kidney diseases, pulmonary hypertension or osteoarthritis (OA), or the GREM1 related diseases are associated with an increased level of GREM1 and are selected from the group consisting of: scleroderma, idiopathic pulmonary fibrosis, diabetic nephropathy, IgAN, lupus nephritis, Alport syndrome, glioma, head and neck cancer, prostate cancer, lung cancer, gastric cancer, pancreatic cancer, esophageal cancer, bladder cancer, breast cancer and colorectal cancer.

In some embodiments, the medicament further comprises a second therapeutic agent, wherein the second therapeutic agent is selected from the group consisting of: chemotherapy drugs e.g., cisplatin), radiotherapeutic drugs, immunotherapeutic drugs (e.g., immuncheckpoint modulators, such as PD-1/PD-L1 axis inhibitors and TGF-β inhibitors), anti-angiogenesis drugs (e.g., antagonists of VEGFR-1, VEGFR-2 and VEGFR-3), targeted therapeutic drugs, cell therapeutic drugs, gene therapeutic drugs, hormone therapeutic drugs, cytokines, and the like.

### BRIEF DESCRIPTION OF THE DRAWINGS

FIG. 1 shows protein stability data measured by SEC in a pH screening experiment using an acetate buffer system, a histidine buffer system and a citrate buffer system respectively.

FIG. 2 shows protein stability data measured by CEX in a pH screening experiment using an acetate buffer system, a histidine buffer system and a citrate buffer system respectively.

FIG. 3 shows protein stability data measured by SEC in an excipient screening experiment using NaCl, arginine hydrochloride, sucrose, sorbitol, trehalose and mannitol respectively as a stabilizer.

FIG. 4 shows protein stability data measured by CEX in an excipient screening experiment using arginine hydrochloride and sucrose respectively as a stabilizer and methionine as an antioxidant in a histidine-histidine hydrochloride buffer system under different pH conditions.

FIG. 5 shows protein stability data measured by CEX in an excipient screening experiment using arginine hydrochloride and sucrose respectively as a stabilizer and methionine as an antioxidant in a histidine-histidine hydrochloride buffer system under different pH conditions.

FIG. 6 shows protein stability data measured by SEC in an excipient screening experiment using arginine hydrochloride and sucrose respectively as a stabilizer and methionine as an antioxidant in a histidine-histidine hydrochloride buffer system under different pH conditions.

FIG. 7 shows protein stability data measured by NR CE-SDS in an excipient screening experiment using arginine hydrochloride and sucrose respectively as a stabilizer and methionine as an antioxidant in a histidine-histidine hydrochloride buffer system under different pH conditions.

FIG. 8 shows protein stability data of the target formulation measured by SEC under long-term experimental conditions at 5° C.

FIG. 9 shows protein stability data of the target formulation measured by NR CE-SDS under long-term experimental conditions at 5° C.

FIG. 10 shows protein stability data of the target formulation measured by CEX under long-term experimental conditions at 5° C.

FIG. 11 shows protein stability data of the target formulation measured by SEC under accelerated experimental conditions at 25° C.

FIG. 12 shows protein stability data of the target formulation measured by NR CE-SDS under accelerated experimental conditions at 25° C.

FIG. 13 shows protein stability data of the target formulation measured by CEX under accelerated experimental conditions at 25° C.

FIG. 14 shows protein stability data of the target formulations measured by CEX under experimental conditions at 40° C.

### DETAILED DESCRIPTION

The following descriptions of the present disclosure are merely intended to describe various embodiments of the present disclosure. The specific examples described shall not be construed as limitations of the scope of the present disclosure. Various equivalent substitutions, alterations or variations may be made by persons skilled in the art without departing from the spirit and essence of the present disclosure, and it shall be understood that all these equivalent embodiments are also included herein. All documents cited herein, including publications, patents and patent applications, are incorporated herein by reference in their entirety. When the method referred to in the disclosure includes two or more qualified steps, the steps defined may be performed in any order or simultaneously (unless the possibility is precluded in the context). Moreover, the method may include one or more of other steps, which may be performed before any steps defined, between two steps defined or after all the steps defined (unless the possibility is precluded in the context).

The reference to “about” (a value or a range of values) herein includes examples of the value or the range of values. For example, “about X” includes “X”. In general, the term “about” refers to the value of a variable, all values that are within an experimental error range of the variable (e.g., within 95% confidence interval of a mean) or all values that are within 10% of the variable, based on greater values. For example, “about X” includes “110%×X”, “109%×X”, “108%×X”, “107%×X”, “106%×X”, “105%×X”, “104%×X”, “103%×X”, “102%×X”, “101%×X”, “99%×X”, “98%×X” “97%×X”, “96%×X”, “95%×X”, “94%×X”, “93%×X”, “92%×X”, “91%×X” or “90%×X”.

The term “at least” and a number thereafter are used herein for indicating the beginning of a range that begins with the number (which may be a range with or without an upper limit, depending on a variable defined). For example, “at least 1” indicates 1 or a value greater than 1. The term “at most” and a number thereafter are used herein for indicating the end of a range that ends with the number (which may be a range with 1 or 0 as a lower limit or a range without a lower limit, depending on a variable defined). For example, “at most 4” indicates 4 or a value less than 4, and “at most 40%” indicates 40% or a value less than 40%. In the present disclosure, when a range is set as “(first number) to (second number)” or “(first number)-(second number)”, it indicates that the lower limit of the range is the first number, and the upper limit is the second number. For example, 5-50 mg/mL indicates a range with 5 mg/mL as a lower limit and 50 mg/mL as an upper limit. The term “less than” or “greater than” a value used herein includes the value.

As used herein, the term “reference antibody” refers to any existing anti-GREM1 antibodies e.g., 6245P) produced on the basis of the sequence of H4H6245P disclosed in WO2014159010, the disclosure of which is incorporated herein by reference in its entirety.

### Antibody

As used herein, the term “antibody” includes any immunoglobulins, monoclonal antibodies, polyclonal antibodies, multivalent antibodies, bivalent antibodies, monovalent antibodies, multispecific antibodies or bispecific antibodies that bind to specific antigens. A complete antibody includes two heavy chains and two light chains. Each heavy chain consists of a heavy chain variable region (V_{H}), a heavy chain first constant region (C_{H1}), a heavy chain second constant region (C_{H2}) and a heavy chain third constant region (C_{H3}). Each light chain consists of a light chain variable region (V_{L}) and a light chain constant region (C_{L}). The V_{H} region of the heavy chain and the V_{L} region of the light chain each has three complementary determinant regions (CDRs), which are interposed between flanking stretches known as framework regions (FR). The framework regions are more highly conserved than the CDRs and forms a scaffold to support the hypervariable loops. The 6 CDRs of one heavy chain and one light chain together constitute an antigen-binding site of an antibody to determine the specificity of the antibody. The antibody described herein also includes fragments or derivatives having an antigen-binding function of a complete antibody. The fragments or the derivatives have the same antigen-binding specificity as the complete antibody, but the binding affinity of the fragments or the derivatives to a specific antigen may be the same as or different from that of the complete antibody.

In some embodiments, the antibody described herein includes an antigen-binding fragment. The antigen-binding fragment refers to one or more types of antibody fragments that retain the binding specificity to an antigen. Examples of the antigen-binding fragment include, but are not limited to, (i) an Fab fragment, which is a monovalent fragment consisting of V_{L}, V_{H}, C_{L} and C_{H1} domains; (ii) an Fab′ fragment, which is an Fab fragment that includes a portion of a hinge region; (iii) an F(ab′)₂ fragment, which is a bivalent fragment containing 2 Fab fragments connected by a disulfide bond in a hinge region; (iv) an Fd fragment consisting of V_{H} and CH₁ domains; (v) an Fv fragment consisting of V_{L} and V_{H} domains of a single arm of an antibody; (vi) a dAb fragment (Ward et al., Nature 341:544-546 (1989); PCT publication WO 90/05144), which comprises a single variable domain; (vii) an isolated CDR; and (viii) a single-stranded Fv fragment, which is a monovalent fragment formed by connection between V_{L} and V_{H} domains directly or via a peptide chain (Huston J S et al., Proc Natl Acad Sci USA, 85:5879(1988)).

In some embodiments, the antibody described herein includes a chimeric antibody with a portion of heavy chain and/or light chain being identical or homologous to corresponding sequences of an antibody that is derived from a particular species or belong to a particular antibody class or subclass, and the remaining portion of the chains being identical or homologous to corresponding sequences of an antibody that is derived from another class or belong to another antibody class or subclass and fragments thereof, provided that it has desired functional activity.

In some embodiments, the antibody described herein includes a humanized antibody. The humanized form of a non-human (e.g., mouse) antibody can be a chimeric immunoglobulin, an immunoglobulin chain or fragments thereof (e.g., Fv, Fab, Fab′, F(ab′)₂ or other antigen-binding sequences of an antibody) that comprises minimal sequences obtained from a non-human immunoglobulin. In some examples, a humanized antibody may be a CDR grafted antibody, wherein amino acid sequences of human CDRs are introduced into amino acid sequences of non-human V_{H} and V_{L} to replace amino acid sequences of corresponding non-human CDRs. In other examples, the majority of the amino acid sequences of a humanized antibody may be derived from a human immunoglobulin (i.e., a receptor antibody), wherein the amino acid residues of CDRs of the receptor antibody are replaced by the amino acid residues of CDRs of a non-human (e.g., mouse, rat and rabbit) antibody having desired specificity, affinity and capability. Usually, a humanized antibody comprises at least one, generally two, variable domains, wherein all or substantially all of the CDR sequences are from a non-human immunoglobulin, and all or substantially all of framework region (FR) sequences are from a human immunoglobulin. In some examples, residues in the framework regions of variable regions of a human immunoglobulin are replaced by corresponding non-human residues. Moreover, a humanized antibody may include residues that exist neither in the original antibody nor in the sequences of introduced CDRs or framework regions.

The anti-GREM1 antibody described herein refers to an antibody that can specifically bind to the gremlin1 (GREM1) protein, such as humanized 14E3 (hzd 14E3), such as Hu14E3_HaLa, Hu14E3_HaLb, Hu14E3_HbLa, Hu14E3_HbLb, Hu14E3_HcLa and Hu14E3_HcLb.

The “Hu14E3_HaLa” described herein refers to humanized 14E3, which comprises a heavy chain variable region having an amino acid sequence as set forth in SEQ ID NO: 7 and a light chain variable region having an amino acid sequence as set forth in SEQ ID NO: 8. In some embodiments, the “Hu14E3_HaLa” described herein further comprises a heavy chain constant region having an amino acid sequence as set forth in SEQ ID NO: 33 and a light chain constant region having an amino acid sequence as set forth in SEQ ID NO: 34. In some embodiments, the “Hu14E3_HaLa” described herein comprises a heavy chain having an amino acid sequence as set forth in SEQ ID NO: 9 and a light chain having an amino acid sequence as set forth in SEQ ID NO: 10.

The “Hu14E3_HaLb” described herein refers to humanized 14E3, which comprises a heavy chain variable region having an amino acid sequence as set forth in SEQ ID NO: 7 and a light chain variable region having an amino acid sequence as set forth in SEQ ID NO: 13. In some embodiments, the “Hu14E3_HaLb” described herein further comprises a heavy chain constant region having an amino acid sequence as set forth in SEQ ID NO: 33 and a light chain constant region having an amino acid sequence as set forth in SEQ ID NO: 34.

The “Hu14E3_HbLa” described herein refers to humanized 14E3, which comprises a heavy chain variable region having an amino acid sequence as set forth in SEQ ID NO: 11 and a light chain variable region having an amino acid sequence as set forth in SEQ ID NO: 8. In some embodiments, the “Hu14E3_HbLa” described herein further comprises a heavy chain constant region having an amino acid sequence as set forth in SEQ ID NO: 33 and a light chain constant region having an amino acid sequence as set forth in SEQ ID NO: 34.

The “Hu14E3_HbLb” described herein refers to humanized 14E3, which comprises a heavy chain variable region having an amino acid sequence as set forth in SEQ ID NO: 11 and a light chain variable region having an amino acid sequence as set forth in SEQ ID NO: 13. In some embodiments, the “Hu14E3_HbLb” described herein further comprises a heavy chain constant region having an amino acid sequence as set forth in SEQ ID NO: 33 and a light chain constant region having an amino acid sequence as set forth in SEQ ID NO: 34.

The “Hu14E3_HcLa” described herein refers to humanized 14E3, which comprises a heavy chain variable region having an amino acid sequence as set forth in SEQ ID NO: 12 and a light chain variable region having an amino acid sequence as set forth in SEQ ID NO: 8. In some embodiments, the “Hu14E3_HcLa” described herein further comprises a heavy chain constant region having an amino acid sequence as set forth in SEQ ID NO: 33 and a light chain constant region having an amino acid sequence as set forth in SEQ ID NO: 34.

The “Hu14E3_HcLb” described herein refers to humanized 14E3, which comprises a heavy chain variable region having an amino acid sequence as set forth in SEQ ID NO: 12 and a light chain variable region having an amino acid sequence as set forth in SEQ ID NO: 13. In some embodiments, the “Hu14E3_HcLb” described herein further comprises a heavy chain constant region having an amino acid sequence as set forth in SEQ ID NO: 33 and a light chain constant region having an amino acid sequence as set forth in SEQ ID NO: 34.

The term “gremlin1” or “GREM1” refers to a variant 1 of gremlin and covers gremlin1 in different species, such as humans, mice, monkeys, and the like. The GREM1 is an evolutionarily conserved and human gremlin1 gene (hGREM1) has been mapped to chromosome 15q13-q15 (Topol L Z, et al., (1997) Molecular Cell Biology (Mol. Cell Biol.), 17: 4801-4810; Topol L Z, et al., Cytogenetic Cell Genetics (Cytogenet Cell Genet.), 89: 79-84). The amino acid sequence of hGREM1 may be accessed in a gene bank database through a deposit number of NP-037504 or a Uniprot database through a deposit number of 060565. The term “human gremlin1” and the term “hGREM1” may be used interchangeably in the present disclosure.

In some embodiments, the anti-GREM1 antibody described herein comprises a heavy chain variable region (V_{H}), wherein the heavy chain variable region (V_{H}) comprises a CDR1 as set forth in SEQ ID NO: 1, a CDR2 as set forth in SEQ ID NO: 2 and/or a CDR3 as set forth in SEQ ID NO: 3.

In some embodiments, the anti-GREM1 antibody described herein comprises a light chain variable region (V_{L}), wherein the light chain variable region (V_{L}) comprises a CDR1 as set forth in SEQ ID NO: 4, a CDR2 as set forth in SEQ ID NO: 5 and/or a CDR3 as set forth in SEQ ID NO: 6.

In some embodiments, the anti-GREM1 antibody described herein comprises a heavy chain variable region (V_{H}) and a light chain variable region (V_{L}), wherein the heavy chain variable region (V_{H}) comprises a CDR1 having an amino acid sequence as set forth in SEQ ID NO: 1, a CDR2 having an amino acid sequence as set forth in SEQ ID NO: 2 and/or a CDR3 having an amino acid sequence as set forth in SEQ ID NO: 3, and the light chain variable region (V_{L}) comprises a CDR1 having an amino acid sequence as set forth in SEQ ID NO: 4, a CDR2 having an amino acid sequence as set forth in SEQ ID NO: 5 and/or a CDR3 having an amino acid sequence as set forth in SEQ ID NO: 6. In some embodiments, the anti-GREM1 antibody comprises a heavy chain CDR1 (HCDR1) as set forth in SEQ ID NO: 1, an HCDR2 as set forth in SEQ ID NO: 2 and an HCDR3 as set forth in SEQ ID NO: 3, as well as a light chain CDR1 (LCDR1) as set forth in SEQ ID NO: 4, an LCDR2 as set forth in SEQ ID NO: 5 and an LCDR3 as set forth in SEQ ID NO: 6. In some embodiments, the anti-GREM1 antibody comprises a CDR1, a CDR2 and a CDR3 of the heavy chain variable region as set forth in SEQ ID NO: 7, as well as a CDR1, a CDR2 and a CDR3 of the light chain variable region as set forth in SEQ ID NO: 8.

In some embodiments, the anti-GREM1 antibody described herein comprises a heavy chain framework region 1 (HFR1) as set forth in QX₁QLVQSGSELKKPGASVKVSCKASGX₂TFT (SEQ ID NO: 24), an HFR2 as set forth in WMX₃QAPGQGLX₄WMG (SEQ ID NO: 25), an HFR3 as set forth in RFX₅FSLDTSVSTAYLQISSLKAEDTAVYYCAR (SEQ ID NO: 26) and an HFR4 as set forth in WGQGTMVTVSS (SEQ ID NO: 17), as well as a light chain framework region 1 (LFR1) as set forth in DVVMTQSPLSLPVTLGQPASISC (SEQ ID NO: 27), an LFR2 as set forth in WLQQRPGQSPRRLIX₆ (SEQ ID NO: 32), an LFR3 as set forth in GVPDRFSGSGSGTDFTLKISRVEAEDVGVYYC (SEQ ID NO: 29) and an LFR4 as set forth in FGQGTKLEIK (SEQ ID NO: 30), wherein X₁ is V or I; X₂ is Y or S; X₃ is R or K; X₄ is E or T; X₅ is V or A; and X₆ is Y or S.

In some embodiments, the anti-GREM1 antibody described herein comprises a heavy chain framework region 1 (HFR1) as set forth in SEQ ID NO: 14, 18 or 21, an HFR2 as set forth in SEQ ID NO: 15, 19 or 22, an HFR3 as set forth in SEQ ID NO: 16, 20 or 23 and an HFR4 as set forth in SEQ ID NO: 17, as well as a light chain framework region 1 (LFR1) as set forth in SEQ ID NO: 27, an LFR2 as set forth in SEQ ID NO: 28 or 31, an LFR3 as set forth in SEQ ID NO: 29 and an LFR4 as set forth in SEQ ID NO: 30. In some embodiments, the anti-GREM1 antibody comprises an FR1, an FR2 and an FR3 of the heavy chain variable region as set forth in SEQ ID NO: 7, 11 or 12, as well as an FR1, an FR2 and an FR3 of the light chain variable region as set forth in SEQ ID NO: 8 or 13.

In some embodiments, the anti-GREM1 antibody described herein comprises a heavy chain variable region (V_{H}) having an amino acid sequence as set forth in SEQ ID NO: 7, 11 or 12. In some embodiments, the anti-GREM1 antibody described herein comprises a light chain variable region (V_{L}) having an amino acid sequence as set forth in SEQ ID NO: 8 or 13. In some embodiments, the anti-GREM1 antibody described herein comprises a heavy chain variable region (V_{H}) having an amino acid sequence as set forth in SEQ ID NO: 7, 11 or 12 and a light chain variable region (V_{L}) having an amino acid sequence as set forth in SEQ ID NO: 8 or 13. In some embodiments, the anti-GREM L antibody described herein comprises a heavy chain variable region as set forth in SEQ ID NO: 7, 11 or 12 and a light chain variable region as set forth in SEQ ID NO: 8 or 13.

In some embodiments, the anti-GREM1 antibody described herein further comprises an immunoglobulin constant region. In some embodiments, the immunoglobulin constant region comprises a heavy chain constant region and/or a light chain constant region. The heavy chain constant region comprises C_{H1}, C_{H1}-C_{H2} or C_{H1}-C_{H3} regions, and the light chain constant region comprises a C_{L} region. In some embodiments, the anti-GREM1 antibody described herein comprises a heavy chain constant region as set forth in SEQ ID NO: 33 and a light chain constant region as set forth in SEQ ID NO: 34.

In some embodiments, the anti-GREM1 antibody described herein comprises a heavy chain having an amino acid sequence as set forth in SEQ ID NO: 9 and a light chain having an amino acid sequence as set forth in SEQ ID NO: 10. In some embodiments, the anti-GREM1 antibody comprises a heavy chain as set forth in SEQ ID NO: 9 and a light chain as set forth in SEQ ID NO: 10.

Exemplary amino acid sequences used in some embodiments are listed in Table A below.

TABLE A
Exemplary amino acid sequences

| SEQ ID | | |
|---|---|---|
| NO. | Amino acid sequence | Name |
| 1 | TYGMA | 14E3-HCDR1 |
| 2 | WINTLSGEPTYADDFKG | 14E3-HCDR2 |
| 3 | EPMDY | 14E3-HCDR3 |
| 4 | KSSQSLLDSDGKTYLS | 14E3-LCDR1 |
| 5 | LVSKLDS | 14E3-LCDR2 |
| 6 | WQGAHFPLT | 14E3-LCDR3 |
| 7 | QVQLVQSGSELKKPGASVKVSCKASGYTFTTYGMAWMR | Hu14E3-Ha VH |
| | QAPGQGLEWMGWINTLSGEPTYADDFKGRFVFSLDTSVST | |
| | AYLQISSLKAEDTAVYYCAREPMDYWGQGTMVTVSS | |
| 11 | QIQLVQSGSELKKPGASVKVSCKASGYTFTTYGMAWMRQ | Hu14E3-Hb VH |
| | APGQGLEWMGWINTLSGEPTYADDFKGRFAFSLDTSVSTA | |
| | YLQISSLKAEDTAVYYCAREPMDYWGQGTMVTVSS | |
| 12 | QIQLVQSGSELKKPGASVKVSCKASGSTFTTYGMAWMKQ | Hu14E3-Hc VH |
| | APGQGLTWMGWINTLSGEPTYADDFKGRFAFSLDTSVSTA | |
| | YLQISSLKAEDTAVYYCAREPMDYWGQGTMVTVSS | |
| 8 | DVVMTQSPLSLPVTLGQPASISCKSSQSLLDSDGKTYLSWL | Hu14E3-La VL |
| | QQRPGQSPRRLIYLVSKLDSGVPDRFSGSGSGTDFTLKISRV | |
| | EAEDVGVYYCWQGAHFPLTFGQGTKLEIK | |
| 13 | DVVMTQSPLSLPVTLGQPASISCKSSQSLLDSDGKTYLSWL | Hu14E3-Lb VL |
| | QQRPGQSPRRLISLVSKLDSGVPDRFSGSGSGTDFTLKISRV | |
| | EAEDVGVYYCWQGAHFPLTFGQGTKLEIK | |
| 9 | QVQLVQSGSELKKPGASVKVSCKASGYTFTTYGMAWMR | Hu14E3-Ha HC |
| | QAPGQGLEWMGWINTLSGEPTYADDFKGRFVFSLDTSVST | |
| | AYLQISSLKAEDTAVYYCAREPMDYWGQGTMVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDKTHTCPPCPAPELLGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKC | |
| | KVSNKALPAPIEKTISKAKGQPREPQVYTLPPSRDELTKNQ | |
| | VSLTCLVKGFYPSDIAVEWESNGQPENNYKTTPPVLDSDG | |
| | SFFLYSKLTVDKSRWQQGNVFSCSVMHEALHNHYTQKSL | |
| | SLSPGK | |
| 10 | DVVMTQSPLSLPVTLGQPASISCKSSQSLLDSDGKTYLSWL | Hu14E3-La LC |
| | QQRPGQSPRRLIYLVSKLDSGVPDRFSGSGSGTDFTLKISRV | |
| | EAEDVGVYYCWQGAHFPLTFGQGTKLEIKRTVAAPSVFIF | |
| | PPSDEQLKSGTASVVCLLNNFYPREAKVQWKVDNALQSG | |
| | NSQESVTEQDSKDSTYSLSSTLTLSKADYEKHKVYACEVT | |
| | HQGLSSPVTKSFNRGEC | |
| 33 | ASTKGPSVFPLAPSSKSTSGGTAALGCLVKDYFPEPVTVSW | Heavy chain |
| | NSGALTSGVHTFPAVLQSSGLYSLSSVVTVPSSSLGTQTYIC | constant region |
| | NVNHKPSNTKVDKKVEPKSCDKTHTCPPCPAPELLGGPSV | of human IgG1 |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVYTLPPSRDELTK | |
| | NQVSLTCLVKGFYPSDIAVEWESNGQPENNYKTTPPVLDS | |
| | DGSFFLYSKLTVDKSRWQQGNVFSCSVMHEALHNHYTQK | |
| | SLSLSPGK | |
| 34 | RTVAAPSVFIFPPSDEQLKSGTASVVCLLNNFYPREAKVQW | Light chain |
| | KVDNALQSGNSQESVTEQDSKDSTYSLSSTLTLSKADYEK | constant region |
| | HKVYACEVTHQGLSSPVTKSFNRGEC | of human κ |

TABLE B
Exemplary amino acid sequences in framework regions (FRs) of hzd
14E3 (hu14E3)

| | | FR1 | FR2 | FR3 | FR4 |
|---|---|---|---|---|---|
| hu14E3 | Ha | SEQ ID NO: 14 | SEQ ID NO: 15 | SEQ ID NO: 16 | SEQ ID NO: |
| | | QVQLVQSGSE | WMRQAPGQG | RFVFSLDTSVST | 17 |
| | | LKKPGASVKV | LEWMG | AYLQISSLKAE | WGQGTMVTV |
| | | SCKASGYTFT | | DTAVYYCAR | SS |
| | Hb | SEQ ID NO: 18 | SEQ ID NO: 19 | SEQ ID NO: 20 | SEQ ID NO: |
| | | QIQLVQSGSEL | WMRQAPGQG | RFAFSLDTSVST | 17 |
| | | KKPGASVKVS | LEWMG | AYLQISSLKAE | WGQGTMVTV |
| | | CKASGYTFT | | DTAVYYCAR | SS |
| | Ho | SEQ ID NO: 21 | SEQ ID NO: 22 | SEQ ID NO: 23 | SEQ ID NO: |
| | | QIQLVQSGSEL | WMKQAPGQG | RFAFSLDTSVST | 17 |
| | | KKPGASVKVS | LTWMG | AYLQISSLKAE | WGQGTMVTV |
| | | CKASGSTFT | | DTAVYYCAR | SS |
| | General | SEQ ID NO: 24 | SEQ ID NO: 25 | SEQ ID NO: 26 | |
| | formula | QX₁QLVQSGSE | WMX₃QAPGQ | RFX₅FSLDTSVS | |
| | | LKKPGASVKV | GLX₄WMG | TAYLQISSLKA | |
| | | SCKASGX₂TFT | | EDTAVYYCAR | |
| hu14E3 | La | SEQ ID NO: 27 | SEQ ID NO: 28 | SEQ ID NO: 29 | SEQ ID NO: |
| | | DVVMTQSPLS | WLQQRPGQSP | GVPDRFSGSGS | 30 |
| | | LPVTLGQPASI | RRLIY | GTDFTLKISRVE | FGQGTKLEIK |
| | | SC | | AEDVGVYYC | |
| | Lb | SEQ ID NO: 27 | SEQ ID NO: 31 | SEQ ID NO: 29 | SEQ ID NO: |
| | | DVVMTQSPLS | WLQQRPGQSP | GVPDRFSGSGS | 30 |
| | | LPVTLGQPASI | RRLIS | GTDFTLKISRVE | FGQGTKLEIK |
| | | SC | | AEDVGVYYC | |
| | General | | SEQ ID NO: 32 | | |
| | formula | | WLQQRPGQSP | | |
| | | | RRLIX₆ | | |

wherein X₁ is V or I; X₂ is Y or S; X₃ is R or K; X₄ is E or T; X₅ is V or A; and X₆ is Y or S.

The present disclosure relates to a pharmaceutical formulation containing an anti-GREM1 antibody (e.g., the anti-GREM1 antibody described herein). In some embodiments, the concentration of the anti-GREM11 antibody in the pharmaceutical formulation of the present disclosure may be 1 mg/ml-200 mg/ml, 1 mg/ml-190 mg/ml, 10 mg/ml-190 mg/ml, 20 mg/ml-180 mg/ml, 20 mg/ml-170 mg/ml, 20 mg/ml-160 mg/ml, 20 mg/ml-150 mg/ml, 20 mg/ml-140 mg/ml, 20 mg/ml-130 mg/ml, 20 mg/ml-120 mg/ml, 20 mg/ml-110 mg/ml, 20 mg/ml-100 mg/ml, 20 mg/ml-90 mg/ml, 20 mg/ml-80 mg/ml, 20 mg/ml-70 mg/ml, 20 mg/ml-60 mg/ml, 20 mg/ml-50 mg/ml, 20 mg/ml-40 mg/ml or 20 mg/ml-30 mg/ml. In some embodiments, the concentration of the anti-GREM1 antibody is any concentration value in the above-mentioned ranges. For example, as required, the concentration of the anti-GREM1 antibody in the pharmaceutical formulation may be at least 5 mg/ml, at least 10 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml and/or at most 200 mg/ml. In some embodiments, the concentration of the anti-GREM1 antibody in the pharmaceutical formulation is 21 mg/ml. In some embodiments, the concentration of the anti-GREM1 antibody in the pharmaceutical formulation is 33 mg/ml.

### Buffer

The term “buffer” generally refers to a buffered solution that resists changes in pH by the action of its acid-base conjugate components. The “buffer” used herein refers to a compound solution known to be safe when used in a pharmaceutical formulation and maintains or controls the pH of the formulation in a desired range. Acceptable buffers capable of controlling the pH in a range from a mild acidic pH value to a mild alkaline pH value (e.g., a pH value of 4.5-8.0) include, but are not limited to, one or any combination of a succinic acid buffer, a citric acid buffer, a phosphoric acid buffer, an acetic acid buffer, an arginine buffer, a 2-amino-2-hydroxymethyl-1,3-propanediol (TRIS) buffer, a histidine buffer, and the like.

The buffer in the formulation solvent may be prepared using any appropriate method known in the art. In some embodiments, the buffer of the present disclosure may be prepared using specific acid-base ion pairs. In one example, excipients of acid-base ion pairs may be accurately weighed and added into pure water that is about 60% of the volume of a target buffer, uniform mixing is conducted, and then the pH value of a resulting solution is determined. When the pH value deviates from a target value, the pH value may be adjusted with appropriate ion pairs. Then, the solution is diluted with pure water to a target weight or a target volume. Finally, the conductivity, osmotic pressure and pH value of the solution are measured for verification.

The stable pharmaceutical formulation of the present disclosure may comprise a buffer such that the pharmaceutical formulation has a pH value of 4.5-8.0, such as a pH value of 4.5-6.0, 4.5-6.5, 5.7-6.3, 6.0-7.0 or 7.0-8.0. In some embodiments, a suitable buffer is used such that the pharmaceutical formulation has a pH value of 4.5-6.5. In particular, the pH value of the pharmaceutical formulation of the present disclosure may be any pH value in the pH ranges listed above, such as 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0.

Examples of the buffer capable of controlling the pH value of the pharmaceutical formulation in a desired range include an acetic acid buffer, a histidine buffer, a citric acid buffer and other organic acid buffers or inorganic acid buffers. Any one of these buffers may be used alone, or 2 or more of these buffers may be combined for use. Preferably, the pharmaceutical formulation of the present disclosure comprises an acetic acid buffer, a histidine buffer. More preferably, the pharmaceutical formulation of the present disclosure comprises a histidine buffer.

The “acetic acid buffer” refers to a buffer comprising acetate radical ions. The acetic acid buffer may include one or more of acetic acids (e.g., glacial acetic acid), potassium acetate, sodium acetate (e.g., sodium acetate trihydrate), and the like. In some embodiments, the acetic acid buffer is an acetic acid-sodium acetate buffer, such as a glacial acetic acid-sodium acetate trihydrate buffer. In some embodiments, the pH value of the acetic acid buffer may be any pH value in the range of 4.5-6.5, such as 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 or 6.5.

The “histidine buffer” refers to a buffer comprising histidine radical ions. The histidine buffer may include one or more of histidine, histidine hydrochloride (e.g., histidine hydrochloride monohydrate), histidine acetate, histidine phosphate, histidine sulfate, and the like. In some embodiments, the histidine buffer may be a histidine-histidine hydrochloride buffer. In some embodiments, the pH value of the histidine buffer may be any pH value in the range of 4.5-6.5, such as 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 or 6.5.

The “citric acid buffer” refers to a buffer comprising citrate radical ions. The citric acid buffer may include one or more of citric acid, monosodium citrate, disodium citrate, trisodium citrate, monopotassium citrate, dipotassium citrate, tripotassium citrate, sodium chloride, potassium chloride, and the like. In some embodiments, the citric acid buffer is a citric acid-trisodium citrate buffer. In some embodiments, the pH value of the citrate buffer may be any pH value in the range of 4.5-6.5, such as 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 or 6.5.

In some embodiments, the buffer used in the pharmaceutical formulation of the present disclosure is an acetic acid buffer or a histidine buffer, and the pH value of the pharmaceutical formulation of the present disclosure is 4.5-6.5. Without wishing to be bound by theory, it is believed that the acetic acid buffer and the histidine buffer are better than the citric acid buffer. For example, when the buffer of the pharmaceutical formulation of the present disclosure is an acetic acid buffer or a histidine buffer, the anti-GREM1 antibody in the pharmaceutical formulation is more stable (e.g., more stable at high temperature), the force between antibody molecules is a repulsive force rather than an attractive force, and the risk of aggregation of molecules is lower.

The concentration of the buffer described herein refers to the concentration of buffer ions in the buffer. In some embodiments, the concentration of a suitable buffer used in the pharmaceutical formulation of the present disclosure may be 5 mM-50 mM, 10 mM-50 mM, 10 mM-40 mM, 10 mM-30 mM and 10 mM-20 mM. In some embodiments, the concentration of the buffer is any concentration value in the above range. For example, according to requirements, the concentration of the buffer may be 5 mM, at least 10 mM, at least 15 mM, at least 20 mM, at least 25 mM, at least 30 mM, at least 35 mM, at least 40 mM, at least 45 mM and/or at most 50 mM, depending on the specific buffer and the stability required for the pharmaceutical formulation.

In some embodiments, the buffer used in the pharmaceutical formulation of the present disclosure is an acidic acid buffer, such as an acetic acid-sodium acetate buffer, and the concentration may be 5 mM-50 mM. In some embodiments, the concentration of the acidic acid buffer may be 5 mM-50 mM, 5 mM-45 mM, 5 mM-40 mM, 5 mM-35 mM, 5 mM-30 mM, 5 mM-25 mM, 5 mM-20 mM, 5 mM-15 mM and 5 mM-10 mM.

In some embodiments, the buffer used in the pharmaceutical formulation of the present disclosure is a histidine buffer, such as a histidine-histidine hydrochloride buffer, and the concentration may be 5 mM-50 mM. In some embodiments, the concentration of the histidine buffer may be 5 mM-50 mM, 5 mM-45 mM, 5 mM-40 mM, 5 mM-35 mM, 5 mM-30 mM, 5 mM-25 mM, 5 mM-20 mM, 5 mM-15 mM and 5 mM-10 mM.

In some embodiments, the buffer used in the pharmaceutical formulation of the present disclosure is an acidic acid buffer or a histidine buffer, and the concentration is 5 mM-50 mM. In some embodiments, the buffer used in the pharmaceutical formulation of the present disclosure is an acidic acid buffer or a histidine buffer, and the concentration is 10 mM-30 mM. In some embodiments, the buffer used in the pharmaceutical formulation of the present disclosure is an acidic acid buffer or a histidine buffer, and the concentration is about 20 mM. In some embodiments, the buffer used in the pharmaceutical formulation of the present disclosure is a histidine buffer, and the concentration is about 20 mM.

### Stabilizer

As used herein, the term “stabilizer” refers to an agent capable of preventing or reducing the chemical and/or physical instability of a protein of interest when bound to the protein. Examples of the stabilizer include sugars, alcohols, acids, salts, polymers, and the like. Examples of the sugars include glucose, sucrose, trehalose, lactose, glucan, and the like. Examples of the alcohols include sorbitol, and the like. Examples of the acids include citric acid, phosphoric acid, tartaric acid, amino acid, ethylenediamine tetraacetic acid, arginine hydrochloride, and the like. Examples of the salts include sodium sulfate, sodium glutamate, sodium chloride (NaCl), potassium chloride, ammonium acetate and the like. Examples of the polymers comprise polyethylene glycol, povidone, and the like.

In some embodiments, the stabilizer used in the pharmaceutical formulation of the present disclosure is selected from sugars. In some embodiments, the stabilizer used in the pharmaceutical formulation of the present disclosure is selected from alcohols. In some embodiments, the stabilizer used in the pharmaceutical formulation of the present disclosure is not mannitol. In some embodiments, the stabilizer used in the pharmaceutical formulation of the present disclosure is selected from salts. In some embodiments, the stabilizer used in the pharmaceutical formulation of the present disclosure is selected from amino acids. In some embodiments, the stabilizer used in the pharmaceutical formulation of the present disclosure is selected from sucrose, trehalose, sorbitol, NaCl, arginine hydrochloride or combinations thereof.

In some embodiments, the concentration of the stabilizer used in the pharmaceutical formulation of the present disclosure may be 0.5% (w/v)-20% (w/v), 0.5% (w/v)-10% (w/v), 0.5% (w/v)-5% (w/v), 0.5% (w/v)-2.5% (w/v), 0.5% (w/v)-1.5% (w/v), 1% (w/v)-15% (w/v), 1% (w/v)-10% (w/v), 1% (w/v)-5% (w/v), 5% (w/v)-10% (w/v), 2% (w/v)-8% (w/v) or 2% (w/v)-5% (w/v), depending on the specific stabilizer and the stability required for the pharmaceutical formulation. In some embodiments, the concentration of the stabilizer is any concentration value in the above range.

In some embodiments, the stabilizer used in the pharmaceutical formulation of the present disclosure is sucrose, and the concentration of the sucrose in the pharmaceutical formulation may be 0.5% (w/v)-20% (w/v). In some embodiments, the concentration of the sucrose in the pharmaceutical formulation may be 0.5% (w/v)-10% (w/v), 1% (w/v)-10% (w/v), 10% (w/v)-20% (w/v) or 5% (w/v)-15% (w/v). In some embodiments, the concentration of the sucrose in the pharmaceutical formulation is 10% (w/v)-10% (w/v). In some embodiments, the concentration of the sucrose in the pharmaceutical formulation is about 5% (w/v). In some embodiments, the concentration of the sucrose in the pharmaceutical formulation is about 8.2% (w/v). In some embodiments, the concentration of the sucrose in the pharmaceutical formulation is about 9% (w/v).

In some embodiments, the stabilizer used in the pharmaceutical formulation of the present disclosure is arginine hydrochloride, and the concentration of the arginine hydrochloride in the pharmaceutical formulation may be 0.5% (w/v)-20% (w/v). In some embodiments, the concentration of the arginine hydrochloride in the pharmaceutical formulation may be 0.5% (w/v)-10% (w/v), 1-10% (w/v), 10-20% (w/v) or 5-15% (w/v). In some embodiments, the concentration of the arginine hydrochloride in the pharmaceutical formulation is 1% (w/v)-5% (w/v). In some embodiments, the concentration of the arginine hydrochloride in the pharmaceutical formulation is about 3% (w/v).

In some embodiments, the stabilizer used in the pharmaceutical formulation of the present disclosure is trehalose, and the concentration of the trehalose in the pharmaceutical formulation may be 0.5% (w/v)-20% (w/v). In some embodiments, the concentration of the trehalose in the pharmaceutical formulation may be 0.5% (w/v)-10% (w/v), 1% (w/v)-10% (w/v), 10% (w/v)-20% (w/v) or 5% (w/v)-15% (w/v). In some embodiments, the concentration of the trehalose in the pharmaceutical formulation is 5% (w/v)-10% (w/v). In some embodiments, the concentration of the trehalose in the pharmaceutical formulation is about 9% (w/v).

In some embodiments, the stabilizer used in the pharmaceutical formulation of the present disclosure is sorbitol, and the concentration of the sorbitol in the pharmaceutical formulation may be 0.5% (w/v)-20% (w/v). In some embodiments, the concentration of the sorbitol in the pharmaceutical formulation may be 0.5% (w/v)-10% (w/v), 1% (w/v)-10% (w/v), 10% (w/v)-20% (w/v) or 5% (w/v)-15% (w/v). In some embodiments, the concentration of the sorbitol in the pharmaceutical formulation is 2% (w/v)-8% (w/v). In some embodiments, the concentration of the sorbitol in the pharmaceutical formulation is about 5% (w/v).

In some embodiments, the stabilizer used in the pharmaceutical formulation of the present disclosure is NaCl, and the concentration of the NaCl in the pharmaceutical formulation may be 0.5% (w/v)-20% (w/v). In some embodiments, the concentration of the NaCl in the pharmaceutical formulation may be 0.5% (w/v)-10% (w/v), 1% (w/v)-10% (w/v), 10% (w/v)-20% (w/v) or 5% (w/v)-15% (w/v). In some embodiments, the concentration of the NaCl in the pharmaceutical formulation is 0.5% (w/v)-1.5% (w/v). In some embodiments, the concentration of the NaCl in the pharmaceutical formulation is about 0.9% (w/v).

### Surfactant

As used herein, the term “surfactant” refers to organic substances with an amphiphilic structure having both hydrophilicity and hydrophobicity; in other words, the organic substances contains groups with opposite solubility trends, such as oil-soluble hydrocarbon chains and water-soluble ionic groups. Depending on charge of surface active moiety, the surfactant may include anionic surfactants, cationic surfactants and nonionic surfactants.

Exemplary surfactant includes polysorbate e.g., polysorbate 20 or 80), poloxamer e.g., poloxamer 188), Triton, polyethylene glycol, polypropylene glycol and copolymers of ethylene glycol and propylene glycol (e.g., Pluronics, PF68 and the like). In some embodiments, the surfactant used in the pharmaceutical formulation of the present disclosure is selected from polysorbate 20 (also called PS 20 or Tween 20), polysorbate 80 (also called PS 80 or Tween 80), or combinations thereof.

In some embodiments, the concentration of the surfactant used in the pharmaceutical formulation of the present disclosure may be 0.005% (w/v)-0.4% (w/v), 0.01% (w/v)-0.3% (w/v), 0.01% (w/v)-0.2% (w/v), 0.025% (w/v)-0.1% (w/v), 0.05% (w/v)-0.1% (w/v) or 0.01% (w/v)-0.1% (w/v), depending on the specific surfactant and the stability required for the pharmaceutical formulation. In some embodiments, the concentration of the surfactant is any concentration value in the above range.

In some embodiments, the surfactant(s) used in the pharmaceutical formulation of the present disclosure are polysorbate 20 and/or polysorbate 80, and the concentration of the polysorbate 20 and/or the polysorbate 80 in the pharmaceutical formulation may be 0.005% (w/v)-0.4% (w/v). In some embodiments, the concentration of the polysorbate 20 and/or the polysorbate 80 in the pharmaceutical formulation may be 0.01% (w/v)-0.2% (w/v). In some embodiments, the concentration of the polysorbate 20 and/or the polysorbate 80 in the pharmaceutical formulation may be 0.025% (w/v)-0.1% (w/v). In some embodiments, the concentration of the polysorbate 20 and/or the polysorbate 80 in the pharmaceutical formulation may be about 0.05% (w/v).

In some embodiments, the surfactant used in the pharmaceutical formulation of the present disclosure is polysorbate 80. In some embodiments, the concentration of the polysorbate 80 in the pharmaceutical formulation is about 0.05% (w/v).

### Antioxidant

As used herein, the term “antioxidant” refers to substances capable of delaying the oxidation of a pharmaceutical formulation by oxygen. The antioxidant is used for affecting various stages of an auto-oxidation process from different aspects, so as to achieve effects of a reductant, a blocker, a synergist and/or a chelating agent and to provide electrons or effective hydrogen atoms to be accepted by free groups, so that an auto-oxidation chain reaction is interrupted, thus reducing an oxidation reaction of a protein. Examplary antioxidants include, but are not limited to, methionine, cysteine, glutathione, sodium thiosulfate and ascorbic acid. In some embodiments, the antioxidant used in the pharmaceutical formulation of the present disclosure may be methionine.

In some embodiments, the concentration of the antioxidant used in the pharmaceutical formulation of the present disclosure may be 0.01% (w/v)-0.2% (w/v), 0.01% (w/v)-0.1% (w/v) or 0.02% (w/v)-0.06% (w/v), depending on the specific antioxidant and the stability required for the pharmaceutical formulation. In some embodiments, the concentration of the antioxidant is any concentration value in the above range.

In some embodiments, the antioxidant used in the pharmaceutical formulation of the present disclosure is methionine, and the concentration of the methionine in the pharmaceutical formulation is 0.01% (w/v)-0.2% (w/v). In some embodiments, the antioxidant used in the pharmaceutical formulation of the present disclosure is methionine, and the concentration of the methionine in the pharmaceutical formulation is 0.02% (w/v)-0.06% (w/v). In some embodiments, the antioxidant used in the pharmaceutical formulation of the present disclosure is methionine, and the concentration of the methionine in the pharmaceutical formulation is about 0.04% (w/v).

### Other Materials

The pharmaceutical formulation of the present disclosure may further optionally comprise other excipients, such as, but not limited to, an isotonic agent, a diluent, and the like.

The term “isotonic agent” refers to a compound or a composition that provides a drug with an appropriate osmotic tension to prevent net flow of water crossing a cell membrane in contact with the drug. In some embodiments, the pharmaceutical formulation of the present disclosure has the same osmotic pressure as human blood. Suitable isotonic agents include, but are not limited to, glycerol, amino acids or proteins e.g., glycine or albumin), salts e.g., sodium chloride) and sugars e.g., glucose, sucrose and lactose).

The term “diluent” refers to pharmaceutically acceptable reagents that may be used for diluting the pharmaceutical formulation of the present disclosure. Typical diluents include water, normal saline, bacteriostatic agents for injection, pH buffers, sterile salt solutions, Ringer's solutions or glucose solutions.

### Formulation

In one aspect, the present disclosure provides a stable pharmaceutical formulation comprising an anti-GREM1 antibody (e.g., the specific anti-GREM1 antibody provided by the present disclosure), a buffer, a stabilizer and a surfactant. The pharmaceutical formulation has a pH value of 4.5-8.0. In some embodiments, the pH value is 4.5-6.5 so as to achieve adequate stability.

In some embodiments, the pharmaceutical formulation of the present disclosure comprises:
(i) an anti-GREM1 antibody (e.g., the specific anti-GREM1 antibody provided by the present disclosure), where the concentration of the anti-GREM1 antibody is 1 mg/ml-200 mg/ml, preferably 20 mg/ml-40 mg/ml, and more preferably about 21 mg/ml or 33 mg/ml; and/or
(ii) a buffer, where the buffer is preferably an acetic acid buffer or a histidine buffer, more preferably a histidine buffer, and the concentration of the buffer in the pharmaceutical formulation is 5 mmol/L-100 mmol/L, preferably 5 mmol/L-50 mmol/L or 10 mmol/L-30 mmol/L, and more preferably about 20 mmol/L; and/or
(iii) a stabilizer, where the stabilizer is preferably sucrose, trehalose, sorbitol, NaCl or arginine hydrochloride,
   (a) the stabilizer is preferably sucrose, and the concentration of the stabilizer in the pharmaceutical formulation is 0.5% (w/v)-20% (w/v), preferably 1% (w/v)-10% (w/v), and more preferably about 5% (w/v), about 8.2% (w/v) or about 9% (w/v), or
   (b) the stabilizer is preferably arginine hydrochloride, and the concentration of the stabilizer in the pharmaceutical formulation is 0.5% (w/v)-20% (w/v), preferably 1% (w/v)-5% (w/v), and more preferably about 3% (w/v), or
   (c) the stabilizer is preferably trehalose, and the concentration of the stabilizer in the pharmaceutical formulation is 0.5% (w/v)-20% (w/v), preferably 5% (w/v)-10% (w/v), and more preferably about 9% (w/v), or
   (d) the stabilizer is preferably sorbitol, and the concentration of the stabilizer in the pharmaceutical formulation is 0.5% (w/v)-20% (w/v), preferably 2% (w/v)-8% (w/v), and more preferably about 5% (w/v), or
   (e) the stabilizer is preferably NaCl, and the concentration of the stabilizer in the pharmaceutical formulation is 0.5% (w/v)-20% (w/v), preferably 0.5% (w/v)-1.5% (w/v), and more preferably about 0.9% (w/v); and/or
(iv) a surfactant, where the surfactant is preferably polysorbate 80 or polysorbate 20, more preferably polysorbate 80, and the concentration of the surfactant in the pharmaceutical formulation is 0.005% (w/v)-0.4% (w/v), preferably 0.01% (w/v)-0.2% (w/v), more preferably 0.025% (w/v)-0.10% (w/v), and more preferably about 0.05% (w/v); and/or
(v) an antioxidant, where the antioxidant is preferably methionine, cysteine, glutathione, sodium thiosulfate and ascorbic acid, more preferably methionine, and the concentration of the antioxidant in the pharmaceutical formulation is 0.01% (w/v)-0.2% (w/v), preferably 0.02% (w/v)-0.06% (w/v), and more preferably about 0.04% (w/v); and
   the pharmaceutical formulation has a pH value of about 4.5-6.5, preferably about 5.5-6.5.

In some embodiments, the pharmaceutical formulation of the present disclosure comprises an anti-GREM1 antibody (e.g., the specific anti-GREM1 antibody provided by the present disclosure) having a concentration of about 1 mg/ml-200 mg/ml and an acetic acid buffer or a histidine buffer having a concentration of about 5 mmol/L-50 mmol/L, where the pH value of the pharmaceutical formulation is 4.5-6.5. In some embodiments, the pharmaceutical formulation comprises an anti-GREM1 antibody having a concentration of about 20 mg/ml-40 mg/ml. In some embodiments, the pharmaceutical formulation comprises an acetic acid buffer or a histidine buffer having a concentration of about 10 mg/ml-30 mg/ml.

In some embodiments, the pharmaceutical formulation of the present disclosure comprises an anti-GREM1 antibody (e.g., the specific anti-GREM1 antibody provided by the present disclosure) having a concentration of about 1 mg/ml-200 mg/ml, an acetic acid buffer or a histidine buffer having a concentration of about 5 mmol/L-50 mmol/L, and sucrose, trehalose, sorbitol, NaCl or arginine hydrochloride having a concentration of about 0.5% (w/v)-20% (w/v), where the pH value of the pharmaceutical formulation is 4.5-6.5. In some embodiments, the pharmaceutical formulation comprises an anti-GREM1 antibody having a concentration of about 20 mg/ml-40 mg/ml. In some embodiments, the pharmaceutical formulation comprises an acetic acid buffer or a histidine buffer having a concentration of about 10 mg/ml-30 mg/ml. In some embodiments, the pharmaceutical formulation comprises sucrose having a concentration of about 1% (w/v)-10% (w/v). In some embodiments, the pharmaceutical formulation comprises arginine hydrochloride having a concentration of about 1% (w/v)-5% (w/v). In some embodiments, the pharmaceutical formulation comprises trehalose having a concentration of about 5% (w/v)-10% (w/v). In some embodiments, the pharmaceutical formulation comprises sorbitol having a concentration of about 2% (w/v)-8% (w/v). In some embodiments, the pharmaceutical formulation comprises NaCl having a concentration of about 0.5% (w/v)-1.5% (w/v).

In some embodiments, the pharmaceutical formulation of the present disclosure comprises an anti-GREM1 antibody (e.g., the specific anti-GREM1 antibody provided by the present disclosure) having a concentration of about 1 mg/ml-200 mg/ml, an acetic acid buffer or a histidine buffer having a concentration of about 5 mg/ml-50 mmol/L, sucrose, trehalose, sorbitol, NaCl or arginine hydrochloride having a concentration of about 0.5% (w/v)-20% (w/v), and polysorbate 80 or polysorbate 20 having a concentration of about 0.005% (w/v)-0.4% (w/v), where the pH value of the pharmaceutical formulation is 4.5-6.5. In some embodiments, the pharmaceutical formulation comprises an anti-GREM1 antibody having a concentration of about 20 mg/ml-40 mg/ml. In some embodiments, the pharmaceutical formulation comprises an acetic acid buffer or a histidine buffer having a concentration of about 10 mg/ml-30 mg/ml. In some embodiments, the pharmaceutical formulation comprises sucrose having a concentration of about 1% (w/v)-10% (w/v), arginine hydrochloride having a concentration of about 1% (w/v)-5% (w/v), trehalose having a concentration of about 5% (w/v)-10% (w/v), sorbitol having a concentration of about 2% (w/v)-8% (w/v), or NaCl having a concentration of about 0.5% (w/v)-1.5% (w/v). In some embodiments, the pharmaceutical formulation comprises polysorbate 80 or polysorbate 20 having a concentration of about 0.025% (w/v)-0.1% (w/v).

In some embodiments, the pharmaceutical formulation of the present disclosure comprises an anti-GREM1 antibody (e.g., the specific anti-GREM1 antibody provided by the present disclosure) having a concentration of about 1 mg/ml-200 mg/ml, an acetate buffer or a histidine buffer having a concentration of about 5 mmol/L-50 mmol/L, sucrose, trehalose, sorbitol, NaCl or arginine hydrochloride having a concentration of about 0.5% (w/v)-20% (w/v), polysorbate 80 or polysorbate 20 having a concentration of about 0.005% (w/v)-0.4% (w/v), and methionine having a concentration of about 0.01% (w/v)-0.2% (w/v), where the pH value of the pharmaceutical formulation is 4.5-6.5. In some embodiments, the pharmaceutical formulation comprises an anti-GREM1 antibody having a concentration of about 20 mg/ml-40 mg/ml. In some embodiments, the pharmaceutical formulation comprises an acetic acid buffer or a histidine buffer having a concentration of about 10 mg/ml-30 mg/ml. In some embodiments, the pharmaceutical formulation comprises sucrose having a concentration of about 1% (w/v)-10% (w/v), arginine hydrochloride having a concentration of about 1% (w/v)-5% (w/v), trehalose having a concentration of about 5% (w/v)-10% (w/v), sorbitol having a concentration of about 2% (w/v)-8% (w/v), or NaCl having a concentration of about 0.5% (w/v)-1.5% (w/v). In some embodiments, the pharmaceutical formulation comprises polysorbate 80 or polysorbate 20 having a concentration of about 0.025% (w/v)-0.1% (w/v). In some embodiments, the pharmaceutical formulation comprises methionine having a concentration of about 0.02% (w/v)-0.06% (w/v).

In some embodiments, the pharmaceutical formulation of the present disclosure comprises an anti-GREM1 antibody having a concentration of about 20 mg/ml-40 mg/ml, a histidine buffer having a concentration of about 10 mmol/L-30 mmol/L, arginine hydrochloride having a concentration of about 1% (w/v)-10% (w/v), and polysorbate 80 or polysorbate 20 having a concentration of about 0.025% (w/v)-0.1% (w/v), where the pH value of the pharmaceutical formulation is about 4.5-6.5 or about 5.5-6.5.

In some embodiments, the pharmaceutical formulation of the present disclosure comprises an anti-GREM1 antibody having a concentration of about 20 mg/ml-40 mg/ml, a histidine buffer having a concentration of about 10 mg/ml-30 mmol/L, arginine hydrochloride having a concentration of about 1% (w/v)-10% (w/v), polysorbate 80 or polysorbate 20 having a concentration of about 0.025% (w/v)-0.1% (w/v), and methionine having a concentration of about 0.01% (w/v)-0.2% (w/v), where the pH value of the pharmaceutical formulation is about 4.5-6.5 or about 5.5-6.5.

In some embodiments, the pharmaceutical formulation of the present disclosure comprises an anti-GREM1 antibody having a concentration of about 20 mg/ml-40 mg/ml, a histidine buffer having a concentration of about 10 mmol/L-30 mmol/L, sucrose having a concentration of about 1% (w/v)-10% (w/v), and polysorbate 80 or polysorbate 20 having a concentration of about 0.025% (w/v)-0.1% (w/v), where the pH value of the pharmaceutical formulation is about 4.5-6.5 or about 5.5-6.5.

In some embodiments, the pharmaceutical formulation of the present disclosure comprises an anti-GREM1 antibody having a concentration of about 20 mg/ml-40 mg/ml, a histidine buffer having a concentration of about 10 mmol/L-30 mmol/L, sucrose having a concentration of about 1% (w/v)-10% (w/v), polysorbate 80 or polysorbate 20 having a concentration of about 0.025% (w/v)-0.1% (w/v), and methionine having a concentration of about 0.01% (w/v)-0.2% (w/v), where the pH value of the pharmaceutical formulation is about 4.5-6.5 or about 5.5-6.5.

In some embodiments, the pharmaceutical formulation of the present disclosure comprises an anti-GREM1 antibody having a concentration of about 33 mg/ml, a histidine buffer having a concentration of about 20 mmol/L, sucrose having a concentration of about 8% (w/v)-9% (w/v), and polysorbate 80 having a concentration of about 0.05% (w/v), where the pH value of the pharmaceutical formulation is about 5.5-6.5.

In some embodiments, the pharmaceutical formulation of the present disclosure comprises an anti-GREM1 antibody having a concentration of about 33 mg/ml, a histidine buffer having a concentration of about 20 mmol/L, sucrose having a concentration of about 8.2% (w/v), polysorbate 80 having a concentration of about 0.05% (w/v), and methionine having a concentration of about 0.04% (w/v), where the pH value of the pharmaceutical formulation is about 6.0.

The polymer formation due to chemical degradation or aggregation of antibody molecules, or the deglycosylation, glycosylation modification, oxidation of antibody molecules, or other structural modifications that may reduce at least one functional activity of monomer proteins may result in instability of antibody formulations. As for the pharmaceutical formulation comprising an anti-GREM1 antibody, the anti-GREM1 antibody may be chemically degraded during storage of the pharmaceutical formulation, leading to decrease in the concentration of the antibody. The anti-GREM1 antibody may also aggregate to form polymers that are sometimes insoluble in the form of polymeric molecules comprising multiple antibody molecules, leading to decrease in the content of monomers containing single antibody molecules. Therefore, the increase in the content of polymer antibodies will lead to decrease in the purity of monomer antibodies. Moreover, the turbidity of the pharmaceutical formulation may be increased due to the formation of insoluble polymers.

In some embodiments, the pharmaceutical formulation comprising an anti-GREM1 antibody of the present disclosure may still maintain stability after long-term storage, treatment (e.g., storage) at high temperature, room temperature, vibration and/or multiple freezing and thawing cycles, wherein the physical and/or chemical stability and/or the functional activity and the like of the anti-GREM1 antibody remain relatively constant over the time. In some embodiments, the antibody protein concentration, the protein purity, the protein activity, the pH value of the formulation, the osmotic pressure of the formulation, the appearance of the formulation, insoluble particles in the formulation and the like may be used as indicators of the stability of the pharmaceutical formulation. A variety of analytical technologies for determining the stability of proteins are available in the art, which are described in Peptide and Protein Drug Delivery, 247-301, edited by Vincent Lee, Marcel Dekker Inc., New York, New York Press (1991) and Jones, A. Adv. Drug Delivery Rev. 10: 29-90 (1993).

In some embodiments, the stability of the pharmaceutical formulation may be determined by methods known in the art under selected conditions over a selected period of time. Exemplary methods include, but are not limited to, dynamic light scattering (DLS), size-exclusion chromatography (SEC), cation exchange chromatography (CEX), non-reducing capillary electrophoresis (NR CE-SDS), pH value determination, protein concentration (Protein Cone.) determination, visual inspection and microfluidic imaging (MFI).

As shown in examples of the present disclosure, the pharmaceutical formulation comprising an anti-GREM1 antibody provided herein has high stability by visual inspection, such as high stability under long-term storage, high stability at high temperature e.g., 40° C.) or room temperature, high stability after vibration and high stability after freezing and thawing.

In some embodiments, the stable pharmaceutical formulation refers that: in a DLS test for the pharmaceutical formulation, the protein particle size does not change significantly during storage; the KD value is positive, that is to say, the action force between single antibody molecules is a repulsive force, and no aggregation of the single antibody molecules occurs. As shown in examples of the present disclosure, the pharmaceutical formulation comprising the anti-GREM1 antibody provided herein has high stability in a DLS test, such as high stability under long-term storage, high stability at high temperature e.g., 40° C.) or room temperature, high stability after vibration and high stability after freezing and thawing.

In some embodiments, the stable pharmaceutical formulation refers that: in an SEC test for the pharmaceutical formulation, only a small amount of proteins are degraded during storage of the pharmaceutical formulation, and the contents of high polymers or oligomers increase slowly. As shown in examples of the present disclosure, the pharmaceutical formulation comprising the anti-GREM1 antibody provided herein has high stability as shown in an SEC test, such as high stability under long-term storage, high stability at high temperature e.g., 40° C.) or room temperature, high stability after vibration and high stability after freezing and thawing.

In some embodiments, the stable pharmaceutical formulation refers that: in a CEX test of the pharmaceutical formulation, the charge heterogeneity of the pharmaceutical formulation does not change significantly during storage, and the separation degrees of acidic peaks and alkaline peaks does not change significantly. As shown in examples of the present disclosure, the pharmaceutical formulation comprising the anti-GREM1 antibody provided herein has high stability as shown in a CEX test, such as high stability under long-term storage, high stability at high temperature (e.g., 40° C.) and high stability after freezing and thawing.

In some embodiments, the stable pharmaceutical formulation refers that: in an NR CE-SDS test of the pharmaceutical formulation, only a small amount of proteins are degraded during storage of the pharmaceutical formulation, and the contents of high polymers or oligomers increase slowly. As shown in examples of the present disclosure, the pharmaceutical formulation comprising the anti-GREM1 antibody provided herein has high stability as shown in an NR CE-SDS test, such as high stability under long-term storage, high stability at high temperature (e.g., 40° C.) or room temperature, high stability after vibration and high stability after freezing and thawing.

In some embodiments, the stable pharmaceutical formulation refers that: in a visual inspection test of the pharmaceutical formulation, no obvious changes in the appearance of the pharmaceutical formulation during storage are observed, and the pharmaceutical formulation remains as a clear and colorless liquid.

In some embodiments, the stable pharmaceutical formulation refers that: in an MFI test of the pharmaceutical formulation, the contents of sub-visible particles of different particle size ranges do not have a significant increase trend during storage of the pharmaceutical formulation.

In some embodiments, the stable pharmaceutical formulation refers that: in a protein concentration test of the pharmaceutical formulation, the change of the protein concentration in the pharmaceutical formulation is no more than +/−20%, no more than +/−19%, no more than +/−18%, no more than +/−17%, no more than +/−16%, no more than +/−15%, no more than +/−14%, no more than +/−13%, no more than +/−12%, no more than +/−11%, no more than +/−10%, no more than +/−9%, no more than +/−8%, no more than +/−7%, no more than +/−6%, no more than +/−5%, no more than +/−4%, no more than +/−3%, no more than +/−2%, no more than +/−1% or no more than +/−0.5%, where the protein concentration can be determined by an ultraviolet-visible spectrophotometry in accordance with General Rule 0401 of Chinese Pharmacopoeia (2010 edition), Volume III.

### Preparation of Formulations

The present disclosure provides a method for preparing a pharmaceutical formulation, comprising::
(1) providing a formulation solvent and an anti-GREM1 antibody stock solution, wherein the formulation solvent comprises a buffer;
(2) subjecting the anti-GREM1 antibody stock solution to solvent exchange with the formulation solvent to obtain the pharmaceutical formulation described herein. In some embodiments, the solvent exchange refers to buffer replacement, such as buffer replacement by a dialysis method. In some embodiments, the solvent exchange refers to solvent exchange by filtration. In some embodiments, the filtration refers to sterile filtration and/or other filtration methods well known to persons skilled in the art.
(3) adding a required amount of formulation excipient (e.g., a stabilizer, a surfactant and optionally an antioxidant) into the anti-GREM1 antibody stock solution after the solvent exchange, followed by dilution with a buffer to a target concentration.

In some embodiments, the buffer in the formulation solvent is a histidine buffer having a concentration of about 20 mM.

In some embodiments, the stabilizer is sucrose having a concentration of about 8.2% (w/v), the surfactant is polysorbate 80 having a concentration of about 0.05% (w/v), and the antioxidant is methionine having a concentration of about 0.04% (w/v).

In some embodiments, the solvent exchange refers to buffer replacement. In some embodiments, the anti-GREM1 antibody stock solution is subjected to solvent exchange by dialysis with the formulation solvent. For example, a certain volume of a sample may be placed in a dialysis bag (e.g., Snake Skin® dialysis bag), the dialysis bag is sealed and placed in a target buffer with a volume equal to or greater than 100 times, and stirring is conducted continuously to promote replacement. Dialysis is conducted for an appropriate number of times (e.g., 3 times) for an appropriate period of time (e.g., 4 hours, 4 hours and overnight, respectively) under stirring at an appropriate rate (e.g., 300 rpm).

In some embodiments, the solvent exchange refers to solvent exchange by filtration. In some embodiments, the filtration refers to sterile filtration (e.g., through a filter membrane with a pore size of 0.22 micron, with bacteria being retained on the membrane and the anti-GREM1 antibody solution passing through the filter membrane, thus achieving an antibacterial effect), and/or other filtration methods well known to a person skilled in the art.

### Pharmaceutical Composition

In another aspect, the present disclosure provides a pharmaceutical composition, comprising the pharmaceutical formulation described herein and a second therapeutic agent, wherein the second therapeutic agent is selected from the group consisting of: chemotherapeutic drugs (e.g., cisplatin), radiotherapeutic drugs, immunotherapeutic drugs (e.g., immuncheckpoint regulators, e.g., PD-1/PD-L1 axis inhibitors, tgf-β inhibitors), anti-angiogenesis drugs (e.g., antagonists of VEGFR-1, VEGFR-2 and VEGFR-3), targeted therapeutic drugs, cell therapeutic drugs, gene therapeutic drugs, hormone therapeutic drugs, cytokines and the like.

### Application

In another aspect, the present disclosure further provides a method for treating diseases in a subject in need thereof, comprising administrating a therapeutically effective amount of the pharmaceutical formulation or the pharmaceutical composition provided herein to a subject, where the subject has or is suspicious of having diseases that require treatment with an antibody against GREM1.

As used herein, the term “treatment” refers to reducing or relieving disease conditions or the severity and/or duration of one or more of the symptoms thereof, inhibiting or preventing the progression of the disease conditions, reducing or ending symptoms associated with a condition, and inhibiting or preventing the recurrence, development, onset or progression of one or more of the symptoms associated with the disease conditions. The subjects in need thereof include subjects having had the diseases.

The term “therapeutically effective amount” refers to a measurable minimum concentration required for treating (e.g., improving or preventing) a particular disease condition.

The pharmaceutical formulation or the pharmaceutical composition of the present disclosure can be used for treating GREM1 related diseases, such as chronic diseases and acute diseases. The GREM1 related diseases include cancers, and the like. In some embodiments, the GREM1 related diseases refer to GREM1-expressing cancers. Examples of the GREM1-expressing cancers include, but are not limited to, cancers, fibrotic diseases, angiogenesis, glaucoma or retinal diseases, kidney diseases, pulmonary hypertension or osteoarthritis (OA), or the GREM1 related diseases or disease conditions associated with the increased content of GREM1 and are selected from the following group consisting of: scleroderma, idiopathic pulmonary fibrosis, diabetic nephropathy, IgAN, lupus nephritis, Alport syndrome, glioma, head and neck cancer, prostate cancer, lung cancer, gastric cancer, pancreatic cancer, esophageal cancer, bladder cancer, breast cancer and colorectal cancer.

The pharmaceutical formulation or the pharmaceutical composition of the present disclosure can be administered to a subject by any suitable routes. For example, the pharmaceutical formulation can be administered intravenously to a subject.

In another aspect, the present disclosure provides use of the pharmaceutical formulation or the pharmaceutical composition in the manufacture of a medicaments for preventing and/or treating GREM1 related diseases.

In some embodiments, the medicament further comprise a second therapeutic agent, wherein the second therapeutic agent is selected from the group consisting of: chemotherapeutic drugs (e.g., cisplatin), radiotherapeutic drugs, immunotherapeutic drugs (e.g., immuncheckpoint regulators, such as PD-1/PD-L1 axis inhibitors and TGF-$ inhibitors), anti-angiogenesis drugs (e.g., antagonists of VEGFR-1, VEGFR-2 and VEGFR-3), targeted therapeutic drugs, cell therapeutic drugs, gene therapeutic drugs, hormone therapeutic drugs, cytokines, and the like.

### EXAMPLES

### Summary of Experimental Methods:

In the following embodiments, formulation screening experiments were carried out on formulations of the anti-GREM1 antibody. The anti-GREM1 antibody used in the following examples is Hu14E3_HaLa. Since other humanized anti-GREM1 antibodies e.g., Hu14E3_HaLb, Hu14E3_HbLa, Hu14E3_HbLb, Hu14E3_HcLa and Hu14E3_HcLb) have substantially the same amino acid sequence as Hu14E3_HaLa, it may be expected that the following formulation screening experiments are also applicable to these humanized anti-GREM1 antibodies. In other words, the above humanized anti-GREM1 antibodies (e.g., Hu14E3_HaLa, Hu14E3_HaLb, Hu14E3_HbLa, Hu14E3_HbLb, Hu14E3_HcLa and Hu14E3_HcLb) can also achieve the expected high stability in the formulations provided herein (e.g., high stability under long-term storage, high stability at high temperature (e.g., 40° C.) or room temperature, high stability after vibration and high stability after freezing and thawing). The anti-GREM1 antibody of the present disclosure is prepared by the following method: HEK293E cells having a concentration of 1×10⁶ cells/ml were cultured in a Freestyle 293 expression culture medium comprising 10% of Pluronic F-68 and were transfected with an equal amount of heavy chain vector DNA and light chain vector DNA having a final concentration of 0.5 μg/ml and PEI (polyethyleneimine-straight chain, Polyscience) having a concentration of 1.0 μg/ml. The ratio of the DNA to the PEI was 1:2. A DNA and PEI complex with optimal MEM was formed for 15 minutes at room temperature. The transfected cells were cultured in a flask in 5% of CO₂ at 37° C. at a vibration rate of 125 rpm. 1% of a peptone culture medium was added 22 to 26 hours after the transfection. On the 6^{th} day, the adjusted culture medium was collected, and the supernatant of the adjusted culture medium was subjected to centrifugation at 3,000 rpm for 30 minutes. Then, the clarified adjusted culture medium was loaded onto an nProteinA column (G. E. Healthcare), the column was washed with PBS containing 0.1% of triton-X100, and finally the bound IgG was eluted with a solution containing 0.1 M glycine with a pH value of 3.5. The eluted antibody protein was dialyzed into PBS and stored at −80° C. In order to remove endotoxin, the purified protein was further processed by a Hitrap DEAE Sepharose F.F. Column, and the resulting antibody was analyzed by size-exclusion chromatography (Superdex 200 5/150 GL, GE Healthcare) to determine the purity level. Detailed description may be found in PCT/CN2022/072297, the disclosure of which is incorporated herein by reference in its entirety.

The formulations of the anti-GREM1 antibody tested in the following examples of the present disclosure were prepared by the following methods:

### 1. Preparation of a Target Formulation Solution:

a) A buffer comprising the anti-GREM1 antibody (namely stock solution, also abbreviated as DS in the following examples) was replaced into a target formulation buffer by a dialysis method. Specifically, a certain volume of a DS sample was placed in a Snake Skin® dialysis bag, the dialysis bag was sealed and then placed in a target buffer with a volume equal to or greater than 100 times, and stirring was conducted continuously to promote the replacement. Dialysis was performed for 3 times, for 4 hours, 4 hours and overnight respectively, and the stirring rate was 300 rpm.
b) After the dialysis was completed, a required amount of a stabilizer and a surfactant stock solution were added into the DS, followed by dilution into a target concentration with a target buffer system to obtain a target formulation solution.

The formulations of the anti-GREM1 antibody tested in the following examples of the present disclosure were analyzed by the following analytical methods:

2. Dynamic light scattering (DLS): the protein particle size and the distribution were determined by DLS, and parameters of the method were as follows: collection was conducted for 5 seconds for a total of 20 times for each measurement, and measurement was conducted at a temperature of 25° C.
3. Size-exclusion chromatography (SEC): the protein aggregation was determined by an SEC method with an ultra-high performance liquid chromatography system (UPLC) of Waters and a TSKgelG3000SWXL SEC column (7.8×300 mm, 5 μm) of Tosoh as a chromatographic column. A mobile phase included a 50 mM sodium phosphate buffer and 300 mM NaCl, and the pH value was 6.8+/−0.1. The flow rate was 1.0 mL/min. A sample was diluted to 10 mg/mL and detected at a volume of 10 μL and a wavelength of 280 nm.
4. Cation exchange chromatography (CEX): the charge heterogeneity of proteins was determined by CEX with a Thermo Propac Elite WCX-10 4 mm×150 mm 5 um column in an Agilent 1260 Infinity system. A sample was diluted to 2.00 mg/mL with a mixed solution of a mobile phase A and a mobile phase B.
5. Non-reducing capillary electrophoresis (NR CE-SDS): protein fragments were determined by a CE-SDS (NR) method. A standard sample or a test sample was diluted to 4 mg/mL with a phosphate-citrate buffer, and then 25 μL of the sample was subjected to vortex mixing with 75 μL of an SDS sample buffer and 5 μL of NEM (100 mM N-ethylmaleimide), followed by denaturation treatment. The denatured sample was subjected to centrifugation, followed by incubation at 70±2° C. for 10±2 minutes, cooling at room temperature and centrifugation again. Separation was performed on PA800 plus using an SDS separation gel kit and an uncoated fused quartz capillary tube.
6. Visual inspection: The appearance of a sample was inspected by a YB-2 clarity detector under a black background. The transparency and the color were reported.
7. Microfluidic imaging (MFI): The number of sub-visible particles of specified sizes (2-10 μm, 10-25 μm and a size greater than 25 μm) was determined by microfluidic imaging 5200. In brief, 500 μL of each sample was injected into an MFI instrument according to instructions of an instrument manual. The average number of particles per milliliter was reported.
8. Potency detection: The binding potency of the anti-GREM1 antibody to a Gremlin antigen epitope was detected by an ELISA binding method. The detection was carried out using a human Gremlin/GREM1 protein (Acro) as a coating antigen, a goat anti-human IgG-Fc HRP conjugated (Bethyl laboratories) as a detection antibody and 3,3′,5,5′-tetramethylbenzidine (TMB) as an enzyme reaction substrate. The coating antigen was adsorbed on an ELISA solid phase carrier adsorption plate, washed and sealed. A test sample was added to bind to the coating antigen, followed by incubation and washing. The detection antibody was added, followed by incubation and washing to remove the unbound detection antibody. Then, the substrate was added for color development. Finally, a reaction termination solution was added, and the absorption value was read on a microplate reader at a detection wavelength of 450 nm/650 nm.

### Example 1: PH Screening Experiment

A pH screening test was carried out on formulations of the anti-GREM1 antibody protein. The stability of the anti-GREM1 antibody in an acetic acid buffer system, a histidine buffer system or a citric acid buffer system was investigated in a pH range of 4.5 to 6.5. Specific design of formulations is as shown in Table 1. The stability (tested by SEC, NR CE-SDS, CEX and/or MFI) of all formulations was investigated at the initial time point TO, after storage at 40° C. for 3 days, 5 days and 7 days (40° C. (3D, 5D and 7D)) and after freezing and thawing for 3 cycles and 5 cycles (FT (3C, 5C)).

TABLE 1
Design of a pH screening experiment

| | | Protein | | Stabilily |
|---|---|---|---|---|
| Formu- | | concentration | | conditions and |
| lation | pH | (mg/ml) | Buffer | test items |
| F1 | 4.5 | 21 | 20 mM acetic acid- | T0: SEC, NR |
| | | | sodium acetate | CE-SDS, CEX |
| F2 | 5.0 | | 20 mM acetic acid- | 40° C. (3D, 5D, |
| | | | sodium acetate | 7D): SEC, NR |
| F3 | 5.5 | | 20 mM acetic acid- | CE-SDS, CEX |
| | | | sodium acetate | FT (3C, 5C): |
| F4 | 5.5 | 21 | 20 mM histidine- | SEC, NR CE- |
| | | | histidine | SDS, CEX, MFI |
| | | | hydrochloride | |
| F5 | 6.0 | | 20 mM histidine- | |
| | | | histidine | |
| | | | hydrochloride | |
| F6 | 5.5 | | 20 mM citric acid- | |
| | | | sodium citrate | |
| F7 | 6.0 | | 20 mM citric acid- | |
| | | | sodium citrate | |
| F8 | 6.5 | | 20 mM citric acid- | |
| | | | sodium citrate | |

Experimental results are as follows.

Freezing and thawing (SC): Under the conditions of freezing and thawing for 5 cycles (SC), all the formulations had no significant changes in the protein appearance, protein S concentration, SEC, DLS and NR CE-SDS results. In an MFI experiment, after treatment under freezing and thawing stress for S cycles, the contents of sub-visible particles of different particle size ranges in the formulations F6, F7 and F8 were significantly increased; the stability of the formulations F6, F7 and F8 were significantly worse than those of other formulations. In general, the formulations F1 to F5 had no significant differences (data are as shown in the table below). Therefore, the citric acid-sodium citrate buffer system was not conducive to the storage of the protein at low temperature.

| | | | | Freezing and thawing_3 | | | Freezing and thawing_5 | | |
|---|---|---|---|---|---|---|---|---|---|
| | | | | cycles | | | cycles | | |
| | T0 | | | FT_3x (FT 3C) | | | FT_5x (FT 5C) | | |
| | ≥2 | ≥10 | ≥25 | ≥2 | ≥10 | ≥25 | ≥2 | ≥10 | ≥25 |
| | μm, <10 | μm, <25 | μm, <100 | μm, <10 | μm, <25 | μm, <100 | μm, <10 | μm, <25 | μm, <100 |
| Formulation | μm | μm | μm | μm | μm | μm | μm | μm | μm |
| F1 | 634 | 73 | 15 | 447 | 11 | 0 | 409 | 19 | 4 |
| F2 | 1273 | 34 | 0 | 829 | 8 | 0 | 4479 | 65 | 62 |
| F3 | 768 | 8 | 0 | 795 | 69 | 8 | 1043 | 19 | 4 |
| F4 | 233 | 19 | 0 | 264 | 11 | 0 | 386 | 8 | 0 |
| F5 | 225 | 0 | 0 | 203 | 4 | 0 | 1983 | 19 | 0 |
| F6 | 524 | 42 | 0 | 2920 | 375 | 8 | 65298 | 2794 | 46 |
| F7 | 432 | 15 | 0 | 2610 | 191 | 0 | 63491 | 1907 | 8 |
| F8 | 1330 | 4 | 0 | 424 | 15 | 0 | 60927 | 1494 | 8 |

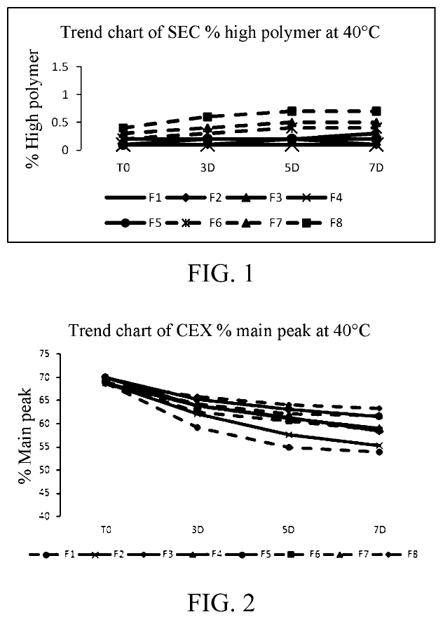

40° C. (3D, 5D, 7D): Under the conditions of 40° C., all the formulations had no significant changes in the protein appearance, protein concentration and DLS results. As shown in FIG. 1, the SEC data show that the contents and increase rates of high molecular weight (HMW) polymers in the formulations F6, F7 and F8 were slightly higher than those of other formulations. As shown in FIG. 2, CEX main peaks of the formulations F1 and F2 were degraded at the highest rate. The purity of main peaks and contents of high polymers of other formulations were not substantially changed, and the formulations had no significant differences among each other.

In conclusion, through investigation of the stability based on freezing and thawing and a temperature of 40° C., the citric acid-sodium citrate buffer system was not conducive to the storage of the protein at low temperature. At 40° C., the increase rates of high molecular weight (HMW) polymers and low molecular weight (LMW) fragments of the formulation F8 in the citric acid-sodium citrate buffer system were slight higher than those of other formulations, and CEX main peaks of the formulations F1 and F2 in the acetic acid-sodium acetate buffer system were degraded at the highest rate. Therefore, the histidine-histidine hydrochloride buffer solution was a preferred buffer system.

### Example 2: Excipient Screening

An excipient screening experiment was carried out on formulations of the anti-GREM1 antibody protein. The protection effects of inorganic salts (e.g., sodium chloride), amino acids (e.g., arginine hydrochloride), alcohols (e.g., sorbitol and mannitol) and sugars (e.g., sucrose and trehalose) on the protein were studied. Specific design of formulations is as shown in Table 2. Samples were tested after incubation. The stability (tested by SEC, NR CE-SDS, CEX and/or MFI) of all formulations was investigated at the initial time point TO, after storage at 40° C. for 3 days, 7 days and 14 days (40° C. (3D), 7D and 14D)) and after freezing and thawing for 1 cycle, 3 cycles and 5 cycles (FT (1C, 3C, 5C)).

TABLE 2

| | | | | | Stabilty |
|---|---|---|---|---|---|
| | Protein | | Concentration | | conditions |
| | concentration | Buffer | of PS80 | | and test |
| Formulation | (mg/mL) | system | (w/v, %) | Excipient | items |
| F9 | 33.0 | 20 mM | 0.05 | 150 mM (namely | T0: SEC, NR |
| | | histidine- | | about 0.88% | CE-SDS, CEX |
| | | histidine | | (w/v)) NaCl | 40° C. (3 D, |
| | | hydrochloride, | | | 7 D, 14 D): |
| F10 | | pH 5.7 | | 150 mM (namely | SEC, NR |
| | | | | about 3.16% | CE-SDS, |
| | | | | (w/v)) | CEX |
| | | | | arginine | FT (1 C, 3 C, |
| | | | | hydrochloride | 5 C): SEC, |
| F11 | | | | 5% (w/v) of | NR CE-SDS, |
| | | | | sucrose | CEX, MFI |
| F12 | | | | 5% (w/v) of | |
| | | | | sorbitol | |
| F13 | | | | 9% (w/v) of | |
| | | | | sucrose | |
| F14 | | | | 9% (w/v) of | |
| | | | | trehalose | |
| F15 | | | | 5% (w/v) of | |
| | | | | mannitol | |

Experimental results are as follows.

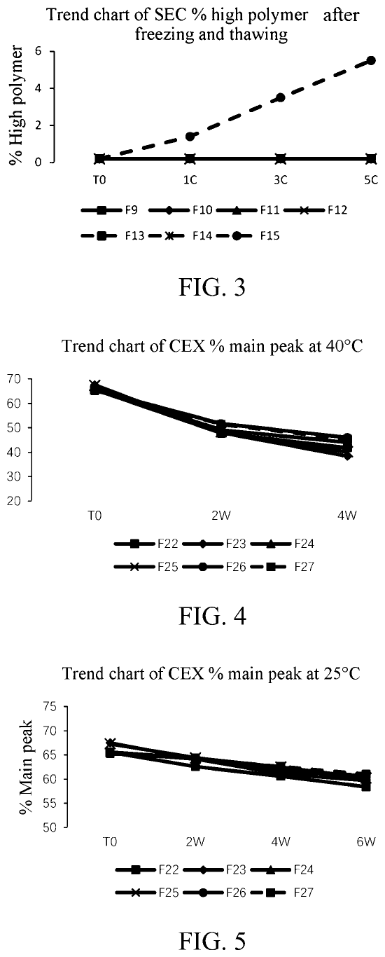

1. Freezing and thawing for 5 cycles: all the formulations had no significant changes in the concentration, CEX results and NR CE-SDS results. The DLS results (as shown in Table 3) showed that the protein in the formulation F15 (mannitol) exhibited as a polydispersed system (PDI %>25%) after treatment under freezing and thawing stress at TO, indicating that the protein in the formulation had a risk of aggregation. During treatment under freezing and thawing stress for 5 cycles on SEC, the formulation F15 had a significantly increased content of HMW polymers (FIG. 3), while other formulations had no significant differences and no changes in main peaks.

TABLE 3

| | | | Freezing and | | Freezing and | | Freezing and | |
|---|---|---|---|---|---|---|---|---|
| | T0 | | thawing_1 | | thawing_3 | | thawing_5 | |
| | Radi- | | cycle FT_1x | | cycles FT_3x | | cycles FT_5x | |
| Formu- | us | PDI | Radius | PDI | Radius | PDI | Radius | PDI |
| lation | (nm) | % | (nm) | % | (nm) | % | (nm) | % |
| F9 | 6.3 | 14.5 | 6.3 | 15.6 | 6.2 | 15.4 | 6.2 | 14.4 |
| F10 | 6.2 | 13.9 | 6.3 | 16.6 | 6.2 | 15.6 | 6.2 | 15.6 |
| F11 | 5.3 | 10.1 | 5.3 | 11.9 | 5.4 | 11.1 | 5.3 | 11.7 |
| F12 | 4.9 | 6.8 | 4.9 | 8.0 | 4.9 | 6.9 | 4.9 | 8.8 |
| F13 | 5.5 | 15.9 | 5.4 | 17.2 | 5.6 | 17.0 | 5.5 | 16.7 |
| F14 | 5.0 | 13.9 | 4.9 | 13.5 | 5.0 | 12.7 | 5.0 | 12.7 |
| F15 | 5.2 | 27.3 | 5.4 | 27.0 | 5.5 | 27.0 | 5.6 | 25.3 |

### 2. Experimental Results at 40° C.:

During the 14-day period under the 40° C. stress, it was observed from the experimental results that: all the formulations had no significant changes in high molecular weight (HMW) polymers; the contents of low molecular weight (LMW) fragments was slightly increased; the CEX main peaks were significant decreased; and there were no significant differences among the formulations (FIG. 14). During the four-week period under the 40° C. incubation, the formulations F10 and F13 were selected for investigation of the oxidation trend. LC-MS results show that: the 2 formulations have a significant oxidation trend at the sites of HC_M101, HC_M109, HC_M249 and HC_M425 (Table 4).

TABLE 4
Oxidation results of an excipient screening study at 40° C.

| | | | 40° C._14 | 40° C._4 |
|---|---|---|---|---|
| | | | days | weeks |
| | | | 40° C._2 | 40° C._4 |
| Oxidation | Formulation | T0 (%) | W (%) | W (%) |
| HC_M101 | F10 | 0.8 | NT | 6.9 |
| | F13 | | 2.7 | 7.2 |
| HC_M109 | F10 | 2.8 | NT | 20.2 |
| | F13 | | 9.3 | 23.6 |
| HC_M249 | F10 | 4.0 | NT | 25.7 |
| | F13 | | 9.8 | 26.0 |
| HC_M425 | F10 | 2.0 | NT | 12.7 |
| | F13 | | 4.8 | 11.5 |

Note:
NT refers to no test.

In conclusion, the freezing-and-thawing and 40° C. stability investigations showed that mannitol was not conducive to the stability of the protein, and there were no significant differences among other stabilizers. Considering that sucrose is widely used in biological formulations and arginine hydrochloride can protect proteins by bonding through hydrogen bonds, the sucrose and the arginine hydrochloride were hence selected for subsequent experiments. In addition, LC-MS results showed that the protein has a significant oxidation trend at some sites, and an antioxidant (i.e., methionine) was added for subsequent formulation screening.

### Example 3: Surfactant Screening

A surfactant screening experiment was carried out on the anti-GREM1 antibody protein. The protection effects of polysorbate 80 and polysorbate 20 having a concentration of 0.025-0.1% (w/v) were studied. Specific designs of formulations are shown in Table 5. The stability (tested by SEC, NR CE-SDS and CEX) of all formulations was investigated at the initial time point TO, after storage at 40° C. for 3 days, 7 days and 14 days (40° C. (3D, 7D and 14D)) and after vibration at 25° C. at a rate of 200 rpm for 1 day, 3 days and 5 days (vibration (200 rpm, 25° C.: 1D, 3D, 5D)).

TABLE 5

| | | | | Surfactant | |
|---|---|---|---|---|---|
| | Protein | | | type and | Stability |
| | concentration | Buffer | Excipient | concentration | conditions and |
| Formulation | (mg/mL) | system | (w/v, %) | (w/v, %) | test items |
| F16 | 33.0 | 20 mM | 9% of | 0.025% of PS80 | T0: SEC, NR |
| F17 | | histidine- | sucrose | 0.05% of PS80 | CE-SDS, CEX |
| F18 | | histidine | | 0.10% of PS80 | 40° C. (3 D, 7 D, |
| F19 | | hydrochloride, | | 0.025% of PS20 | 14 D): SEC, NR |
| F20 | | pH 5.7 | | 0.05% of PS20 | CE-SDS, CEX |
| F21 | | | | 0.10% of PS20 | Vibration (200 |
| | | | | | rpm, 25° C.: 1 D, |
| | | | | | 3 D, 5 D): SEC, |
| | | | | | NR CE-SDS, CEX |

### 1. Analysis of Results of Vibrated Samples:

According to the experimental results, it showed that after vibration for 5 days, all the formulations had no significant changes in high molecular weight (HMW) polymers, low molecular weight (LMW) fragments and charge isomers, and there were significant differences among the formulations.

### 2. Analysis of Results of Samples at 40° C.:

According to the experimental results, it showed that: after incubation at 40° C. for 14 days, all the formulations had no significant changes in high molecular weight (HMW) polymers; the contents of low molecular weight (LMW) fragments were slightly increased; the contents of CEX main peaks were significantly decreased; and there were no significant differences among the formulations.

In conclusion, the vibration and 40° C. stability investigations showed that the polysorbate 80 and the polysorbate 20 having a concentration of 0.025% (w/v)-0.1% (w/v) had good protection effects (with no significant differences between each other) on the protein, both of which met the experimental requirements. 0.05% (w/v) of polysorbate 80 was selected for subsequent studies.

### Example 4: PH Screening and Stabilizer and Antioxidant Experiments

This experiment was carried out to further investigate a histidine-histidine hydrochloride buffer system under different pH conditions, two stabilizers (arginine hydrochloride and sucrose) and the antioxidant effect of methionine. Specific designs of formulations are shown in Table 6. The stability (tested by SEC, NR CE-SDS, CEX, visual inspection (visible particles) and/or MFI and potency detection) of all formulations were investigated at the initial time point TO, after storage at 40° C. for 2 weeks and 4 weeks (40° C. (2W, 4W)), after storage at 25° C. for 2 weeks, 4 weeks and 6 weeks (25° C. (2W, 4W, 6W)), after storage at 5° C. for 2 weeks, 4 weeks, 6 weeks and 3 months (5° C. (2W, 4W, 6W, 3M)), after freezing and thawing for 1 cycle, 3 cycles and 5 cycles (FT (1C, 3C, 5C)), after vibration at 25° C. at a rate of 1,000 rpm for 8 hours (vibration (1,000 rpm, 25° C.: 8 h)), and after stirring at 25° C. at a rate of 400 rpm for 1 hour (stirring (400 rpm, 25° C.: 1 h)).

TABLE 6

| | Protein | | Surfactant | | Stability |
|---|---|---|---|---|---|
| | concentration | Buffer | concentration | | conditions |
| Formulation | (mg/mL) | system | (w/v, %) | Excipient | and test items |
| F22 | 33.0 | 20 mM | 0.05% of | 150 mM (namely | T0: SEC, NR |
| | | histidine- | polysorbate | about 3.16% | CE-SDS, CEX |
| | | histidine | 80 | (w/v)) arginine | 40° C. (2 W, |
| | | hydrochloride, | | hydrochloride | 4 W): SEC, NR |
| F23 | | pH 5.7 | | 9% of sucrose | CE-SDS, |
| F24 | | 20 mM | | 150 mM (namely | CEX, potency |
| | | histidine- | | about 3.16% | 25° C. (2 W, |
| | | histidine | | (w/v)) arginine | 4 W, 6 W): |
| | | hydrochloride, | | hydrochloride | SEC, NR CE- |
| F25 | | pH 6.0 | | 9% of sucrose | SDS, CEX |
| F26 | | | | 150 mM (namely | 5° C. (2 W, 4 W, |
| | | | | about 3.16% | 6 W, 3 M): |
| | | | | (w/v)) arginine | SEC, NR CE- |
| | | | | hydrochloride and | SDS, CEX, |
| | | | | 0.04% of | visual |
| | | | | methionine | inspection |
| F27 | | 20 mM | | 150 mM (namely | (visible |
| | | histidine- | | about 3.16% | particles) |
| | | histidine | | (w/v)) arginine | FT (1 C, 3 C, |
| | | hydrochloride, | | hydrochloride | 5 C): SEC, NR |
| | | pH 6.5 | | | CE-SDS, |
| | | | | | CEX, MFI |
| | | | | | Vibration |
| | | | | | (1,000 rpm, |
| | | | | | 25° C.: 8 h): |
| | | | | | SEC, NR CE- |
| | | | | | SDS, CEX |
| | | | | | Stirring (400 |
| | | | | | rpm, 25° C.: |
| | | | | | 1 h): SEC, NR |
| | | | | | CE-SDS, CEX |

### 1. Freezing and Thawing, Vibration and Stirring Results:

According to the experimental results, it was observed that under the conditions of freezing and thawing, vibration and stirring, all the formulations had no significant changes in high molecular weight (HMW) polymers, low molecular weight (LMW) fragments and charge isomers, and there were no significant differences among the formulations.

### 2. Lighting Results:

According to the experimental results, it was observed that: all the formulations had no significant changes in high molecular weight (HMW) polymers and low molecular weight (LMW) fragments; the contents of CEX main peaks were slightly decreased; and there were no significant differences among the formulations.

### 3. 40° C. Results:

According to the experimental results, it was observed that: at 40° C., all the formulations had slight increased contents of high molecular weight (HMW) polymers, significantly increased contents of low molecular weight (LMW) fragments and decreased contents of CEX main peaks (FIG. 4); there were no significant differences among the formulations. In addition, according to potency detection results, there were no significant differences among the formulations (a TO sample was used as a reference sample in the potency detection). As shown in Table 7, after incubation at a high temperature of 40° C. for 4 weeks, the binding potency results of all formulations were within an acceptable standard range (which is 50-150% of the potency of the reference sample). As shown in Table 8, after comparing the liquid chromatography-mass spectrometry (LC-MS) data of the formulations F24 and F26, it was observed that the methionine had a significant antioxidant effect.

TABLE 7

| Formulation | 40° C._4 W_(%) |
|---|---|
| F22 | 73% |
| F23 | 75% |
| F24 | 72% |
| F25 | 81% |
| F26 | 84% |
| F27 | 79% |

TABLE 8

| | | | 40° C._2 | 40° C._4 |
|---|---|---|---|---|
| Oxidation | Formulation | T0 (%) | W_(%) | W_(%) |
| HC_M101 | F24 | NT | 6.0 | 5.7 |
| | F26 | 1.5 | 2.7 | 3.0 |
| HC_M109 | F24 | NT | 15.6 | 17.1 |
| | F26 | 3.4 | 6.3 | 7.6 |
| HC_M249 | F24 | NT | 22.1 | 17.8 |
| | F26 | 3.7 | 7.9 | 7.7 |
| HC_M425 | F24 | NT | 9.8 | 8.3 |
| | F26 | 2.4 | 4.0 | 4.2 |

Note:
NT refers to no test.

### 25° C. Results:

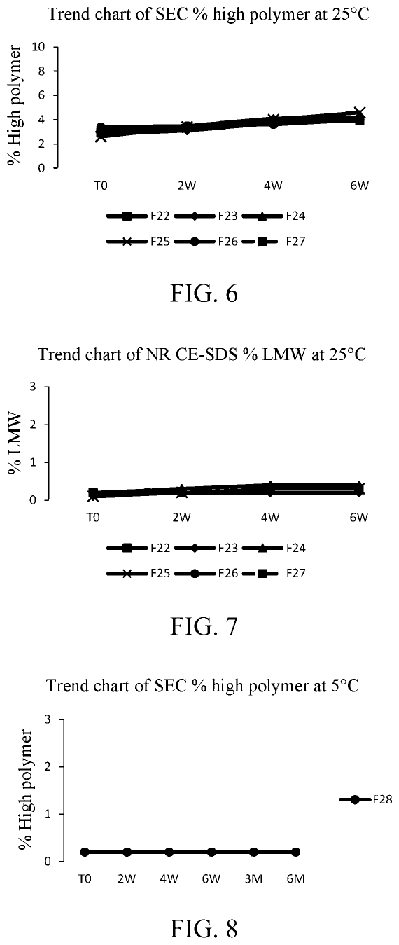

According to the experimental results, it was observed that: at 25° C., all the formulations had no significant changes in high molecular weight (HMW) polymers and low molecular weight (LMW) fragments; the content of CEX main peaks was decreased to some degree; and there were no significant differences among the formulations (FIG. 5 to FIG. 7).

### 5. 5° C. Results:

According to the experimental results, it was observed that: at 5° C., all the formulations had no significant changes in high molecular weight (HMW) polymers, low molecular weight (LMW) fragments and charge isomers; and there were no significant differences among the formulations. However, at 3 months (3M), the formulations F22, F24, F26 and F27 (namely all the formulations comprising arginine hydrochloride) had visible particles (Table 9), while the formulations F23 and F25 comprising sucrose had no visible particles.

TABLE 9

| | Appearance |
|---|---|
| Formulation | 3M |
| F22 | UPS |
| F23 | NVP |
| F24 | UPS |
| F25 | NVP |
| F26 | UPS |
| F27 | UPS |

Note:
UPS refers to an uncountable particle swarm; and NVP refers to no visible particles.

Considering the results under all conditions comprehensively, all the formulas PGP-32′n containing arginine hydrochloride had visible particles after placement at 5° C. for 3 months. Thus, sucrose is a preferred stabilizer. In order to make the osmotic pressure of the formulations similar to that of human plasma as much as possible, the concentration of the sucrose was adjusted from 9% (w/v) to 8.2% (w/v). The histidine-histidine hydrochloride buffer systems (including the formulas 22, 24 and 27) with different pH values (pH 5.7, pH 6.0 and pH 6.5) had no obvious differences. Thus, the middle pH, namely pH 6.0, is selected as a preferred pH value. The methionine has an obvious antioxidant effect. Therefore, a preferred formula comprises 20 mM histidine/histidine hydrochloride, 8.2% (w/v) of sucrose, 0.04% (w/v) of methionine and 0.05% (w/v) of polysorbate 80 and has a pH value of 6.0 for a formula verification study.

### Example 5: Formulation Verification Experiment

This experiment was carried out to further investigate the stability of the preferred formulation selected in Example 4 under various conditions. The preferred formulation comprises 20 mM histidine/histidine hydrochloride, 8.200 (w/v) of sucrose, 0.04% (w/v) of methionine and 0.05% (w/v) of polysorbate 80 and having a pH value of 6.0. Specific designs of formulations are shown in Table 10. The stability (tested by SEC, NR CE-SDS, CEX, visual inspection (visible particles) and/or MFI) of all formulations was investigated at the initial time point T0, after storage at 40° C. for 2 weeks and 4 weeks (40° C. (2W, 4W)), after storage at 25° C. for 2 weeks, 4 weeks and 6 weeks (25° C. (2W, 4W, 6W)), after storage at 5° C. for 2 weeks, 4 weeks, 6 weeks, 3 months and 6 months (5° C. (2W, 4W, 6W, 3M, 6M)), after freezing and thawing for 1 cycle, 3 cycles and 5 cycles (FT (C, 3C, 5C)), after vibration at 25° C. at a rate of 200 rpm for 3 days and 7 days (vibration (200 rpm, 25° C.: 3D, 7D)), after stirring at 25° C. at a rate of 400 rpm for 2 hours and 4 hours (400 rpm, 253C: 2H, 4H), and after lighting for 5 days and 10 days.

TABLE 10

| Formulation | Description | Stress conditions and test items |
|---|---|---|
| F28 | 33.0 mg/mL of DS, 20 | 1. The X test and the Y test in the note column were |
| | mM histidine, 8.2% | carried out at the initial time point (T0^{XY}). |
| | (w/v) of sucrose, 0.04% | 2. The X test and the Y test in the note column were |
| | (w/v) of methionine and | carried out after freezing and thawing for 1 cycle, 3 |
| | 0.05% (w/v) of | cycles and 5 cycles (FT (1 C, 3 C, 5 C)^{XY}). |
| | polysorbate 80, pH 6.0 | 3. The X test in the note column was carried out |
| | | after vibration at a rate of 200 rpm for 3 days and 7 |
| | | days (vibration, 200 rpm, 25° C. (3 D, 7 D)^{X}). |
| | | 4. The X test in the note column was carried out |
| | | after lighting treatment for 5 days, and the X test |
| | | and the Z test in the note column were carried out |
| | | after lighting treatment for 10 days (lighting, (5 D, |
| | | 10 D^{Z})^{X}). |
| | | 5. The X test in the note column was carried out |
| | | after lighting control treatment for 5 days and 10 |
| | | days (treatment in a dark place, (5 D, 10 D)^{X}). |
| | | 6. The X test in the note column was carried out |
| | | after stirring at a rate of 400 rpm for 2 hours and 4 |
| | | hours (stirring, 400 rpm, 25° C. (2 H, 4 H)^{X}). |
| | | 7. The X test in the note column was carried out |
| | | after storage at 25° C. for 2 weeks, 4 weeks and 6 |
| | | weeks (25° C., (2 W, 4 W, 6 W)^{X}). |
| | | 8. The X test in the note column was carried out |
| | | after storage at 5° C. for 2 weeks, 4 weeks, 6 weeks, |
| | | 3 months and 6 months (5° C., (2 W, 4 W, 6 W, 3 M, |
| | | 6 M)^{X}). |
| | | 9. The X test in the note column was carried out |
| | | after storage at 40° C. for 2 weeks and 4 weeks, and |
| | | the Z test in the note column was also carried out |
| | | on the samples at 4 weeks (40° C. (2 W, 4 W^{Z}))^{X} |

X refers to concentration, appearance, NR CE-SDS, SEC or CEX; Y refers to MFI; and Z refers to potency.

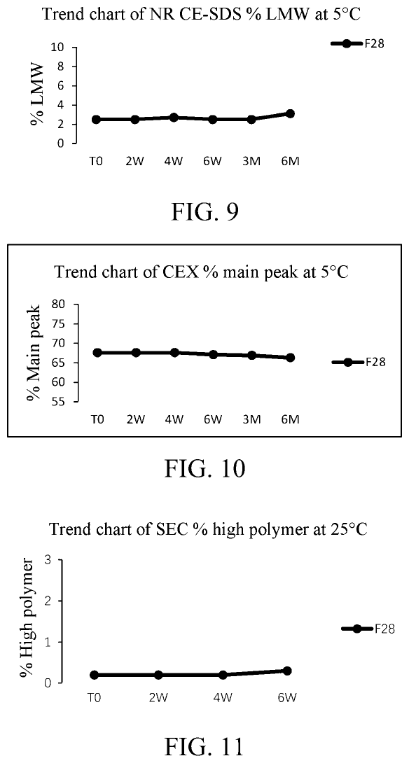

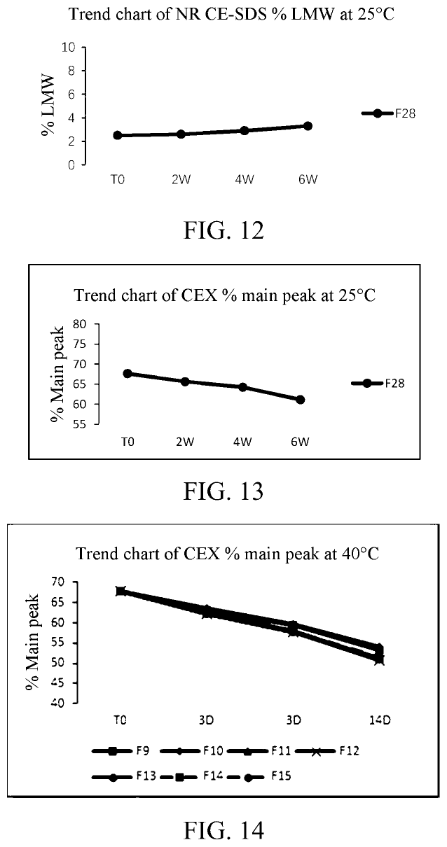

According to the experimental results, it was observed that: the final formulation had good stability under the experimental conditions of long-term storage at 5° C. (FIG. 8 to FIG. 10), accelerated storage at 25° C. (FIG. 11 to FIG. 13), freezing and thawing, vibration and stirring; and various test indicators were not significantly changed (Table 11 to Table 14). Under lighting conditions, although the CEX main peaks were significantly decreased, the binding potency results of the formulation were within an acceptable standard range (which is 500%-150% of the potency of the reference sample). Thus, the potency was not significantly changed, and the requirements for stability were met. At 40° C. (Table 15), although the LMW was significantly increased and the CEX main peaks were significantly decreased, the binding potency results of the formulation after 4 weeks were within an acceptable standard range (which is 50%-150% of the potency of the reference sample). Thus, the potency was not significantly changed. Therefore, the selected buffer system and formulation components can provide good stability for the GREM11 protein.

TABLE 11
Freezing and thawing results

| Test items | | T0 | 1x | 3x | 5x |
|---|---|---|---|---|---|
| SEC | % HMW | 0.2 | 0.2 | 0.2 | 0.2 |
| | % MP | 98.7 | 98.8 | 98.7 | 98.8 |
| | % LMW | 1.2 | 1.0 | 1.0 | 1.0 |
| NR CE-SDS | % LMW | 2.5 | 2.5 | 2.5 | 2.5 |
| | % MP | 97.2 | 97.3 | 97.3 | 97.3 |
| | % HMW | 0.3 | 0.2 | 0.2 | 0.2 |
| CEX | % MP | 67.6 | 67.7 | 67.7 | 67.7 |
| | % Acid Peak | 16.8 | 16.8 | 16.7 | 16.7 |
| | % Basic Peak | 15.6 | 15.6 | 15.6 | 15.6 |
| MFI | ≥2 μm, <10 μm | 1429 | 528 | 1028 | 830 |
| | ≥10 μm, <25 μm | 109 | 0 | 35 | 4 |
| | ≥25 μm, <100 μm | 16 | 0 | 4 | 0 |

TABLE 12
Stirring results

| Test items | | T0 | 400 rpm, 2 H | 400 rpm, 4 H |
|---|---|---|---|---|
| SEC | % HMW | 0.2 | 0.2 | 0.2 |
| | % MP | 98.7 | 98.8 | 98.9 |
| | % LMW | 1.2 | 1.1 | 0.9 |
| NR CE-SDS | % LMW | 2.5 | 2.5 | 2.6 |
| | % MP | 97.2 | 97.3 | 97.1 |
| | % HMW | 0.3 | 0.1 | 0.2 |
| CEX | % MP | 67.6 | 67.5 | 67.6 |
| | % Acid Peak | 16.8 | 16.8 | 16.8 |
| | % Basic Peak | 15.6 | 15.6 | 15.6 |

TABLE 13
Vibration results

| Test items | | T0 | 3 D | 7 D |
|---|---|---|---|---|
| SEC | % HMW | 0.2 | 0.2 | 0.2 |
| | % MP | 98.7 | 98.8 | 98.7 |
| | % LMW | 1.2 | 1.0 | 1.1 |
| NR CE-SDS | % LMW | 2.5 | 2.6 | 2.7 |
| | % MP | 97.2 | 97.2 | 97.2 |
| | % HMW | 0.3 | 0.2 | 0.1 |
| CEX | % MP | 67.6 | 67.1 | 66.7 |
| | % Acid Peak | 16.8 | 16.9 | 17.0 |
| | % Basic Peak | 15.6 | 16.0 | 16.3 |

TABLE 14
Lighting and lighting control

| | | Lighting (lighting | | | Lighting control (treatment | | |
|---|---|---|---|---|---|---|---|
| | | at 25° C.) | | | in a dark place at 25° C.) | | |
| Test items | | T0 | 5 D | 10 D | T0 | 5 D | 10 D |
| SEC | % HMW | 0.2 | 0.3 | 0.4 | 0.2 | 0.2 | 0.2 |
| | % MP | 98.7 | 98.5 | 98.3 | 98.7 | 98.8 | 98.7 |
| | % LMW | 1.2 | 1.2 | 1.3 | 1.2 | 1.0 | 1.0 |
| NR | % LMW | 2.5 | 2.7 | 3.0 | 2.5 | 2.6 | 2.7 |
| CE- | % MP | 97.2 | 97.1 | 96.7 | 97.2 | 97.2 | 96.9 |
| SDS | % HMW | 0.3 | 0.2 | 0.3 | 0.3 | 0.2 | 0.4 |
| CEX | % MP | 67.6 | 60.2 | 53.5 | 67.6 | 66.8 | 66.3 |
| | % Acid Peak | 16.8 | 20.8 | 25.8 | 16.8 | 16.9 | 17.2 |
| | % Basic Peak | 15.6 | 19.0 | 20.7 | 15.6 | 16.3 | 16.5 |

| | | | | | | |
|---|---|---|---|---|---|---|
| Potency | 100% | NT | 86% | NT | NT | NT |

Note:
NT refers to no test.

TABLE 15
40° C.

| Test items | | T0 | 2 W | 4 W |
|---|---|---|---|---|
| SEC | % HMW | 0.2 | 0.5 | 0.6 |
| | % MP | 98.7 | 97.0 | 96.1 |
| | % LMW | 1.2 | 2.5 | 3.3 |
| NR CE-SDS | % LMW | 2.5 | 4.6 | 5.9 |
| | % MP | 97.2 | 94.9 | 94 |
| | % HMW | 0.3 | 0.5 | 0.1 |
| CEX | % MP | 67.6 | 49.4 | 39.9 |
| | % Acid Peak | 16.8 | 28.8 | 37.6 |
| | % Basic Peak | 15.6 | 21.8 | 22.5 |
| Potency | | 100% | NT | 84% |

Note:
NT refers to no test.

### Example 6: Potency Characterization of the Anti-GREM1 Antibody Used in the Present Disclosure

### Analysis of the Binding Affinity Between the Anti-GREM1 Antibody and Human GREM1 Through ELISA

A transparent polystyrene culture plate (BEAVER) was coated with a high pH coating buffer containing 0.5 μg/ml hGREM1 (ACRO) and mouse gremlin (R&D) at 100 μl/well at 4° C. overnight. Then, the culture plate was washed once with PBS and 0.1% of Tween 20 (Sigma) on an automatic culture plate washer. 100 μl of a blocking solution consisting of PBS, 1% of BSA, 1% of standard goat serum and 0.50% of Tween 20 (Sigma) was added into each well for incubation at room temperature for 2 hours. Then, 100 μl of an antibody (started with 2 μg/ml and continuously diluted) in an antibody dilution buffer containing PBS, 1% of BSA, 1% of standard goat serum and 0.01% of Tween 20 was added into each well of the culture plate for incubation at room temperature for 1 hour. Then, the culture plate was washed with 200 μl of a solution containing PBS and 0.1% of Tween 20 for three times, and goat anti-mouse IgG-HRP (Abcam) at a ratio of 1:10000 was added at 100 μl/well for incubation at room temperature for 1 hour. Then, the culture plate was washed with a solution containing PBS and 0.1% of Tween 20 for 3 times. Finally, TMB (Pierce) was added into each well at 100 μl/well, and several minutes later, 50 μl of a termination solution was added into each well. The culture plate was read on a Multiscan FC microplate reader (Thermo Scientific) at 450 nM. As shown in Table 16 and Table 17, compared with a reference antibody 6245P, the anti-GREM1 antibody used in the present disclosure and other humanized anti-GREM1 antibodies (e.g., Hu14E3_HaLb, Hu14E3_HbLa, Hu14E3_HbLb, Hu14E3_HcLa and Hu14E3_HcLb) have better binding affinity with human GREM1.

### Determination of the Affinity of a Humanized Antibody by Fortebio

The human GREM1 protein was diluted to a concentration of 2 μg/ml with a kinetic buffer. The concentration of 0 nM was used as a reference control. The antibody to be tested was diluted to a concentration of 100 nM, 50 nM and 25 nM with a ForteBio kinetic buffer (including PBS with a pH value of 7.4, 0.1% of BSA and 0.002% of Tween-20). Human GREM1-his was fixed to an NTA biosensor. Detection was carried out on a baseline for 60 seconds, and association of the anti-GREM1 antibody was detected for 120 seconds to obtain Kₒₙ factor data. Then, dissociation was performed in a kinetic buffer for 90 seconds to obtain K_{off} factor data. As shown in Table 16, the anti-GREM1 antibody used in the present disclosure has a KD value less than 1 nM, which was significantly lower than the KD value of the reference antibody. That is to say, compared with the reference antibody 6245P, the anti-GREM1 antibody used in the present disclosure had better binding affinity with human GREM1.

### Detection of the Ability of the Anti-GREM1 Antibody to Block the Binding of GREM1 to Human BMP2/4/7 by ELISA

A culture plate was coated with recombinant human BMP2/4 (0.5 μg/ml) overnight, serial diluention buffers were incubated with 1 μg/ml modified human GREM1-his at room temperature for 1 hour, and then a resulting complex was added to the coated culture plate and incubated at room temperature for another one hour. Then, the culture plate was washed, and anti-his HRP (GenScript) was added. Then, the culture plate was developed with a TMB solution, and the development was stopped by adding a termination solution. The culture plate was read on a microplate reader at 450 nm. According to the results shown in Table 16, it indicated that compared with the reference antibody 6245P, the an-GREM1 antibody provided herein could better inhibit the binding of GREM1 to BMP2 or BMP4. That is to say, compared with the reference antibody 6245P, the anti-GREM1 antibody used in the present disclosure had better potency of blocking the binding of human GREM1 to BMP2 or BMP4.

TABLE 16

| | | Anti-GREM1 antibody | Reference |
|---|---|---|---|
| | | used in the present | antibody |
| Potency parameter | | disclosure | 6245P |
| EC50 (ng/ml) | Human GREM1 | 6.90 | 57.5 |
| KD (M) | Human GREM1 | <1.0E−12 | 2.58E−09 |
| IC50 (μg/ml) | BMP2 | 0.4232 | 0.7971 |
| | BMP4 | 0.5551 | 0.6377 |

TABLE 17
EC50 of binding to hGremlin-his

| Antibody | | EC50 (ng/ml) |
|---|---|---|
| 14E3 (hIgG1) | Hu14E3_HaLa | 6.9 |
| | Hu14E3_HaLb | 5.6 |
| | Hu14E3_HbLa | 6.0 |
| | Hu14E3_HbLb | 6.2 |
| | Hu14E3_HcLa | 6.6 |
| | Hu14E3_HcLb | 6.1 |
| | Chimeric 14E3 | 6.8 |
| 6245P (hIgG4) | | 57.5 |

### Efficacy of a Combination of the Anti-GREM1 Antibody of the Present Disclosure and Cisplatin in an Esophageal Cancer PDX Model

Human gremlin IHC specifically positive esophageal tumor tissue (E7) was obtained from Beijing Cancer Hospital passage in NOD/SCID mice and established PDX bank. After testing, it was found that the esophageal cancer PDX model E7 was positive in GREM1 expression but did not have PD-L1 expression.

Each mouse was subcutaneously inoculated with a small tumor tissue block approximately 3 mm in diameter which sheared from integrated tumor decollement form a tumor bearing mouse. 18 days after inoculation animals with tumor size at about 70 mmn³ were selected and randomly divided into 4 groups, each group consisting of 8 mice. Then the mice were treated with isotype control+PBS, the anti-GREM1 antibody of the present disclosure at dose of 20 mg/kg, Cisplatin at dose of 3 mg/kg and combination of the anti-GREM1 antibody of the present disclosure and Cisplatin. Isotype control and the anti-GREM1 antibody of the present disclosure were administrated twice a week for 4 weeks by i.p. injection and PBS, while Cisplatin was administrated once a week for 4 weeks by i.v. injection. Animals were sacrificed at the end of the study with CO₂ inhalation. Tumor size was measured twice or triple times a week in two dimensions using a caliper (INSIZE) and the volume was expressed in mmA3 using the formula: V=0.5 a*b² where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

Table 18 showed significantly enhanced tumor growth inhibition when the anti-GREM1 antibody of the present disclosure alone was used in this experiment as compared to Isotype control. Combination of anti-GREM1 antibody of the present disclosure and Cisplatin further inhibited tumor growth when compared to either anti-GREM1 antibody of the present disclosure alone (63.97% TGI vs 42.92% TGI) or Cisplatin alone (63.97% TGI vs 59.79% TGI), suggesting synergistic effect of the combination treatment with anti-GREM1 antibody of the present disclosure and Cisplatin on esophageal cancer.

To date, first-line therapy for esophageal cancer generally includes esophagectomy, chemotherapy, targeted therapy, immunotherapy (e.g., targeting PD-1 or PD-L1), and/or a combination thereof. Second-line and subsequent therapy for esophageal cancer may involve targeted therapy, such as ramucirumab to target vascular endothelial growth factor (VEGF) receptor or trastuzumab for metastatic adenocarcinoma that overexpresses HER2 (NCCN Clinical Practice Guidelines in Oncology. Esophageal and Esophagogastric Junction Cancers. National Comprehensive Cancer Network. V1. 2020). Our data described above show that the anti-GREM1 antibodies provided herein could effectively treat tumors that do not express PD-L1, e.g., esophageal cancer that do not overexpress PD-L1, and could further achieve synergistic effect when in combination with chemotherapy, e.g., Cisplatin. This suggested that the anti-GREM1 antibodies provided herein can serve as a new option for either first-line therapy or second-line therapy for esophageal cancer.

TABLE 18
Tumor growth inhibition of a combination of the anti-GREM1
antibody used in the present disclosure and cisplatin in an
esophageal cancer PDX model on the 26^{th} day

| | Tumor size (mm³, | | p value in comparison |
|---|---|---|---|
| Treatment (n = 8) | mean +/− S.E.M) | TGI (%) | with isotype control |
| Isotype control at a dose of 20 | 1192.40 +/− 203.22 | / | / |
| mg/kg and PBS | | | |
| Anti-GREM1 antibody used | 680.59 +/− 163.73 | 42.92 | 0.0701 |
| in the present disclosure at a | | | |
| dose of 20 mg/kg | | | |
| Cisplatin at a dose of 3 mg/kg | 479.41 +/− 104.05 | 59.79 | 0.0075 |
| Anti-GREM1 antibody used | 429.65 +/− 86.99 | 63.97 | 0.0039 |
| in the present disclosure at a | | | |
| dose of 20 mg/kg and | | | |
| cisplatin at a dose of 3 mg/kg | | | |

What is claimed is:

1. A pharmaceutical formulation, comprising an anti-GREM1 antibody and a buffer, wherein the buffer is an acetic acid buffer or a histidine buffer, and the pH value is 4.5-6.5.

2. The pharmaceutical formulation according to claim 1, wherein the concentration of the buffer in the pharmaceutical formulation is 5 mM-50 mM or 10 mM-30 mM.

3. The pharmaceutical formulation according to claim 1 or 2, further comprising a stabilizer.

4. The pharmaceutical formulation according to claim 3, wherein the concentration of the stabilizer in the pharmaceutical formulation is 0.5% (w/v)-20% (w/v) or 0.5% (w/v)-10% (w/v).

5. The pharmaceutical formulation according to claim 3 or 4, wherein the stabilizer is selected from the group consisting of: sucrose, trehalose, sorbitol, NaCl and arginine hydrochloride.

6. The pharmaceutical formulation according to claim 5, wherein
(a) the stabilizer is sucrose, wherein the concentration of the sucrose in the pharmaceutical formulation is 1% (w/v)-10% (w/v); and/or
(b) the stabilizer is arginine hydrochloride, wherein the arginine hydrochloride in the pharmaceutical formulation is 1% (w/v)-5% (w/v); and/or
(c) the stabilizer is trehalose, wherein the concentration of the trehalose in the pharmaceutical formulation is 5% (w/v)-10% (w/v); and/or
(d) the stabilizer is sorbitol, wherein the concentration of the sorbitol in the pharmaceutical formulation is 2% (w/v)-8% (w/v); and/or
(e) the stabilizer is NaCl, wherein the concentration of the NaCl in the pharmaceutical formulation is 0.5% (w/v)-1.5% (w/v).

7. The pharmaceutical formulation according to any one of the preceding claims, further comprising a surfactant.

8. The pharmaceutical formulation according to claim 7, wherein the concentration of the surfactant in the pharmaceutical formulation is 0.005% (w/v)-0.4% (w/v) or 0.01% (w/v)-0.2% (w/v).

9. The pharmaceutical formulation according to claim 7 or 8, wherein the surfactant is selected from the group consisting of: polysorbate 80 and polysorbate 20.

10. The pharmaceutical formulation according to claim 9, wherein
(a) the surfactant is polysorbate 80, and the concentration of the polysorbate 80 and/or the polysorbate 20 in the pharmaceutical formulation is 0.025% (w/v)-0.10% (w/v).

11. The pharmaceutical formulation according to any one of the preceding claims, further comprising an antioxidant.

12. The pharmaceutical formulation according to claim 11, wherein the concentration of the antioxidant in the pharmaceutical formulation is 0.01% (w/v)-0.2% (w/v) or 0.02% (w/v)-0.06% (w/v).

13. The pharmaceutical formulation according to claim 11 or 12, wherein the antioxidant is selected from the group consisting of: methionine, cysteine, glutathione, sodium thiosulfate and ascorbic acid.

14. The pharmaceutical formulation according to any one of the preceding claims, wherein the concentration of the anti-GREM1 antibody in the pharmaceutical formulation is 1 mg/ml-200 mg/ml.

15. The pharmaceutical formulation according to claim 14, wherein the concentration of the anti-GREM1 antibody in the pharmaceutical formulation is 20 mg/ml-40 mg/ml.

16. The pharmaceutical formulation according to any one of the preceding claims, wherein the anti-GREM1 antibody comprises a heavy chain CDR1 (HCDR1) as set forth in SEQ ID NO: 1, an HCDR2 as set forth in SEQ ID NO: 2 and an HCDR3 as set forth in SEQ ID NO: 3, and/or a light chain CDR1 (LCDR1) as set forth in SEQ ID NO: 4, an LCDR2 as set forth in SEQ ID NO: 5 and an LCDR3 as set forth in SEQ ID NO: 6.

17. The pharmaceutical formulation according to any one of the preceding claims, wherein the anti-GREM1 antibody comprises a CDR1, a CDR2 and a CDR3 of the heavy chain variable region as set forth in SEQ ID NO: 7, and/or a CDR1, a CDR2 and a CDR3 of the light chain variable region as set forth in SEQ ID NO: 8.

18. The pharmaceutical formulation according to any one of the preceding claims, wherein the anti-GREM1 antibody comprises a heavy chain framework region 1 (HFR1) as set forth in QX₁QLVQSGSELKKPGASVKVSCKASGX₂TFT (SEQ ID NO: 24), an HFR2 as set forth in WMX₃QAPGQGLX₄WMG (SEQ ID NO: 25), an HFR3 as set forth in RFX₅FSLDTSVSTAYLQISSLKAEDTAVYYCAR (SEQ ID NO: 26) and an HFR4 as set forth in WGQGTMVTVSS (SEQ ID NO: 17), and/or a light chain framework region 1 (LFR1) as set forth in DVVMTQSPLSLPVTLGQPASISC (SEQ ID NO: 27), an LFR2 as set forth in WLQQRPGQSPRRLIX₆ (SEQ ID NO: 32), an LFR3 as set forth in GVPDRFSGSGSGTDFTLKISRVEAEDVGVYYC (SEQ ID NO: 29) and an LFR4 as set forth in FGQGTKLEIK (SEQ ID NO: 30), wherein X₁ is V or I; X₂ is Y or S; X₃ is R or K; X₄ is E or T; X₅ is V or A; and X₆ is Y or S.

19. The pharmaceutical formulation according to claim 18, wherein the anti-GREM1 antibody comprises a heavy chain framework region 1 (HFR1) as set forth in SEQ ID NO: 14, 18 or 21, an HFR2 as set forth in SEQ ID NO: 15, 19 or 22, an HFR3 as set forth in SEQ ID NO: 16, 20 or 23 and an HFR4 as set forth in SEQ ID NO: 17, and/or a light chain framework region 1 (LFR1) as set forth in SEQ ID NO: 27, an LFR2 as set forth in SEQ ID NO: 28 or 31, an LFR3 as set forth in SEQ ID NO: 29 and an LFR4 as set forth in SEQ ID NO: 30.

20. The pharmaceutical formulation according to any one of the preceding claims, wherein the anti-GREM1 antibody comprises an FR1, an FR2, an FR3 and an FR4 of the heavy chain variable region as set forth in SEQ ID NO: 7, 11 or 12, and/or an FR1, an FR2, an FR3 and an FR4 of the light chain variable region as set forth in SEQ ID NO: 8 or 13.

21. The pharmaceutical formulation according to any one of the preceding claims, wherein the anti-GREM1 antibody comprises a heavy chain variable region as set forth in SEQ ID NO: 7, 11 or 12, and/or a light chain variable region as set forth in SEQ ID NO: 8 or 13.

22. The pharmaceutical formulation according to any one of the preceding claims, wherein the anti-GREM1 antibody comprises a heavy chain constant region as set forth in SEQ ID NO: 33, and/or a light chain constant region as set forth in SEQ ID NO: 34.

23. The pharmaceutical formulation according to any one of the preceding claims, wherein the anti-GREM1 antibody comprises a heavy chain as set forth in SEQ ID NO: 9, and/or a light chain as set forth in SEQ ID NO: 10.

24. The pharmaceutical formulation according to any one of the preceding claims, comprising an anti-GREM1 antibody, a buffer, a stabilizer and a surfactant, wherein the buffer is a histidine buffer, the stabilizer is arginine hydrochloride or sucrose, the surfactant is polysorbate 80 or polysorbate 20, and the pH value is about 4.5-6.5.

25. The pharmaceutical formulation according to claim 24, wherein the concentration of the anti-GREM1 antibody in the pharmaceutical formulation is 20 mg/ml-40 mg/ml, the concentration of the histidine buffer in the pharmaceutical formulation is 10 mM-30 mM, the concentration of the arginine hydrochloride or the sucrose in the pharmaceutical formulation is 1% (w/v)-10% (w/v), and/or the concentration of the polysorbate 80 or the polysorbate 20 in the pharmaceutical formulation is 0.025% (w/v)-0.1% (w/v).

26. The pharmaceutical formulation according to claim 25, wherein the anti-GREM1 antibody in the pharmaceutical formulation is about 33 mg/ml, the histidine buffer in the pharmaceutical formulation is about 20 mM, the stabilizer is sucrose, wherein the concentration of the sucrose in the pharmaceutical formulation is about 8% (w/v)-9% (w/v), the surfactant is polysorbate 80, wherein the concentration of the polysorbate 80 in the pharmaceutical formulation is about 0.05% (w/v), and the pH value is about 5.5-6.5.

27. The pharmaceutical formulation according to any one of the preceding claims, further comprising an antioxidant, wherein the antioxidant is methionine.

28. The pharmaceutical formulation according to any one of the preceding claims, wherein the concentration of the methionine in the pharmaceutical formulation is about 0.04% (w/v).

29. The pharmaceutical formulation according to claim 27, wherein the concentration of the anti-GREM1 antibody in the pharmaceutical formulation is about 33 mg/ml, the concentration of the histidine buffer in the pharmaceutical formulation is about 20 mM, the stabilizer is sucrose, wherein the concentration of the sucrose in the pharmaceutical formulation is about 8.2% (w/v), the surfactant is polysorbate 80, wherein the concentration of the polysorbate 80 in the pharmaceutical formulation is about 0.05% (w/v), the concentration of the methionine in the pharmaceutical formulation is about 0.04% (w/v), and the pH value is about 6.0.

30. Use of the pharmaceutical formulation according to any one of claims 1 to 29 in the manufacture of a medicament for preventing and/or treating GREM1 related diseases.

31. The use according to claim 30, wherein the GREM1 related diseases are selected from the group consisting of: cancers, fibrosis diseases, angiogenesis, glaucoma or retinal diseases, kidney diseases, pulmonary hypertension or osteoarthritis (OA), or the GREM1 related diseases are associated with an increased level of GREM1 and are selected from the group consisting of: scleroderma, idiopathic pulmonary fibrosis, diabetic nephropathy, IgAN, lupus nephritis, Alport syndrome, glioma, head and neck cancer, prostate cancer, lung cancer, gastric cancer, pancreatic cancer, esophageal cancer, bladder cancer, breast cancer and colorectal cancer.

32. The use according to claim 30 or 31, wherein the medicament further comprises a second therapeutic agent, wherein the second therapeutic agent is selected from the group consisting of: chemotherapeutic drugs (e.g., cisplatin), radiotherapeutic drugs, immunotherapeutic drugs (e.g., immuncheckpoint modulators, such as PD-1/PD-L1 axis inhibitors and TGF-β inhibitors), anti-angiogenesis drugs (e.g., antagonists of VEGFR-1, VEGFR-2 and VEGFR-3), targeted therapeutic drugs, cell therapeutic drugs, gene therapeutic drugs, hormone therapeutic drugs, cytokines and the like.


SEQUENCE LISTING (34 sequences)

SEQ ID NO: 1 - protein, 5 aa, synthetic construct
TYGMA

SEQ ID NO: 2 - protein, 17 aa, synthetic construct
WINTLSGEPTYADDFKG

SEQ ID NO: 3 - protein, 5 aa, synthetic construct
EPMDY

SEQ ID NO: 4 - protein, 16 aa, synthetic construct
KSSQSLLDSDGKTYLS

SEQ ID NO: 5 - protein, 7 aa, synthetic construct
LVSKLDS

SEQ ID NO: 6 - protein, 9 aa, synthetic construct
WQGAHFPLT

SEQ ID NO: 7 - protein, 114 aa, synthetic construct
QVQLVQSGSELKKPGASVKVSCKASGYTFTTYGMAWMRQAPGQGLEWMGWINTLSGEPTYADDFKGRFVFSLDTSVSTAYLQISSLKAEDTAVYYCAREPMDYWGQGTMVTVSS

SEQ ID NO: 8 - protein, 112 aa, synthetic construct
DVVMTQSPLSLPVTLGQPASISCKSSQSLLDSDGKTYLSWLQQRPGQSPRRLIYLVSKLDSGVPDRFSGSGSGTDFTLKISRVEAEDVGVYYCWQGAHFPLTFGQGTKLEIK

SEQ ID NO: 9 - protein, 444 aa, synthetic construct
QVQLVQSGSELKKPGASVKVSCKASGYTFTTYGMAWMRQAPGQGLEWMGWINTLSGEPTYADDFKGRFVFSLDTSVSTAYLQISSLKAEDTAVYYCAREPMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DVVMTQSPLSLPVTLGQPASISCKSSQSLLDSDGKTYLSWLQQRPGQSPRRLIYLVSKLDSGVPDRFSGSGSGTDFTLKISRVEAEDVGVYYCWQGAHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 114 aa, synthetic construct
QIQLVQSGSELKKPGASVKVSCKASGYTFTTYGMAWMRQAPGQGLEWMGWINTLSGEPTYADDFKGRFAFSLDTSVSTAYLQISSLKAEDTAVYYCAREPMDYWGQGTMVTVSS

SEQ ID NO: 12 - protein, 114 aa, synthetic construct
QIQLVQSGSELKKPGASVKVSCKASGSTFTTYGMAWMKQAPGQGLTWMGWINTLSGEPTYADDFKGRFAFSLDTSVSTAYLQISSLKAEDTAVYYCAREPMDYWGQGTMVTVSS

SEQ ID NO: 13 - protein, 112 aa, synthetic construct
DVVMTQSPLSLPVTLGQPASISCKSSQSLLDSDGKTYLSWLQQRPGQSPRRLISLVSKLDSGVPDRFSGSGSGTDFTLKISRVEAEDVGVYYCWQGAHFPLTFGQGTKLEIK

SEQ ID NO: 14 - protein, 30 aa, synthetic construct
QVQLVQSGSELKKPGASVKVSCKASGYTFT

SEQ ID NO: 15 - protein, 14 aa, synthetic construct
WMRQAPGQGLEWMG

SEQ ID NO: 16 - protein, 32 aa, synthetic construct
RFVFSLDTSVSTAYLQISSLKAEDTAVYYCAR

SEQ ID NO: 17 - protein, 11 aa, synthetic construct
WGQGTMVTVSS

SEQ ID NO: 18 - protein, 30 aa, synthetic construct
QIQLVQSGSELKKPGASVKVSCKASGYTFT

SEQ ID NO: 19 - protein, 14 aa, synthetic construct
WMRQAPGQGLEWMG

SEQ ID NO: 20 - protein, 32 aa, synthetic construct
RFAFSLDTSVSTAYLQISSLKAEDTAVYYCAR

SEQ ID NO: 21 - protein, 30 aa, synthetic construct
QIQLVQSGSELKKPGASVKVSCKASGSTFT

SEQ ID NO: 22 - protein, 14 aa, synthetic construct
WMKQAPGQGLTWMG

SEQ ID NO: 23 - protein, 32 aa, synthetic construct; identical to SEQ ID NO: 20

SEQ ID NO: 24 - protein, 30 aa, synthetic construct
  VAR_SEQ 2: note = X is V or I
  VAR_SEQ 27: note = X is Y or S
QXQLVQSGSELKKPGASVKVSCKASGXTFT

SEQ ID NO: 25 - protein, 14 aa, synthetic construct
  VAR_SEQ 3: note = X is R or K
  VAR_SEQ 11: note = X is E or T
WMXQAPGQGLXWMG

SEQ ID NO: 26 - protein, 32 aa, synthetic construct
  VAR_SEQ 3: note = X is V or A
RFXFSLDTSVSTAYLQISSLKAEDTAVYYCAR

SEQ ID NO: 27 - protein, 23 aa, synthetic construct
DVVMTQSPLSLPVTLGQPASISC

SEQ ID NO: 28 - protein, 15 aa, synthetic construct
WLQQRPGQSPRRLIY

SEQ ID NO: 29 - protein, 32 aa, synthetic construct
GVPDRFSGSGSGTDFTLKISRVEAEDVGVYYC

SEQ ID NO: 30 - protein, 10 aa, synthetic construct
FGQGTKLEIK

SEQ ID NO: 31 - protein, 15 aa, synthetic construct
WLQQRPGQSPRRLIS

SEQ ID NO: 32 - protein, 15 aa, synthetic construct
  VAR_SEQ 15: note = X is Y or S
WLQQRPGQSPRRLIX

SEQ ID NO: 33 - protein, 330 aa, synthetic construct [sequence omitted]

SEQ ID NO: 34 - protein, 107 aa, synthetic construct [sequence omitted]